(19) United States
(12) Patent Application Publication
(10) Pub. No.: US 2021/0155812 A1
(43) Pub. Date: May 27, 2021

(54) SILK INK COMPOSITIONS AND METHODS OF MAKING AND USING THE SAME

(71) Applicant: TRUSTEES OF TUFTS COLLEGE, Medford, MA (US)

(72) Inventors: Fiorenzo G. OMENETTO, Lexington, MA (US); Giuseppina MATZEU, Medford, MA (US); Laia MOGAS SOLDEVILA, Cambridge, MA (US)

(73) Assignee: Trustees of Tufts College, Medford, MA (US)

(21) Appl. No.: 17/044,259

(22) PCT Filed: Mar. 29, 2019
(86) PCT No.: PCT/US2019/024953

§ 371 Date: Sep. 30, 2020

Related U.S. Application Data
(60) Provisional application No. 62/650,950, filed on Mar. 30, 2018.

(51) Int. Cl. C09D 11/04; C09D 11/30; C09D 11/52; C09D 105/04; C09D 189/00; A01N 25/08; B01L 3/00; G01N 21/80; G01N 21/78; B41M 1/12

(52) U.S. Cl. CPC C09D 11/04; C09D 11/30; C09D 11/52; C09D 105/04; C09D 189/00; B01L 2200/12; B01L 3/502707; G01N 21/80; G01N 21/78; B41M 1/12; A01N 25/08

(57) ABSTRACT

The present disclosure provides biologically-based ink compositions, methods of making the biologically-based ink composition, as well as articles, objects, devices, and/or apparatuses fabricated from or that comprise the biologically-based ink compositions. The biologically-based ink composition can include a silk fibroin solution having a concentration of silk fibroin between 0.1 wt % and 10 wt %, as well as a thickening agent and a humectant dispersed throughout the silk fibroin solution. The biologically-based ink compositions may be used to functionalize a substrate to fabricate sensors, non-toxic conductive inks/textiles, microfluidic channels, technical apparel or fashion accessories, functionalized furniture, tensile canopies, architectural wall paper, facade components, or may be patterned on a substrate to encapsulate scents, flavors, dyes and pigments, therapeutic agents, or biologically active molecules.

### CROSS-REFERENCE TO RELATED APPLICATIONS

This application claims the benefit of U.S. Provisional Application No. 62/650,950 filed Mar. 30, 2018. The contents of that application are incorporated by reference for all purposes as if set forth in their entirety herein.

### GOVERNMENT SUPPORT

This invention was made with government support under grant W911QY-15-2-0001 awarded by the United States Army and grant N00014-16-1-2437 awarded by the United States Navy. The government has certain rights in the invention.

### BACKGROUND

Screen-printing and inkjet printing are two techniques that are simple, robust, and allow contemporary deposition and patterning of inks onto substrates.

Screen-printing is based on the use of a designed mesh masked with a pattern transferred to the underlying substrate by applying pressure with a squeegee that releases the ink through the designed mesh. It is a robust, fast, and scaled-up process for mass production, however, significant technological gaps are still observable in many production phases, especially in the development and post-processing of the screen-printed materials (e.g., UV curing, temperature annealing in the range of 120-180° C.). Recent research on screen-printing inks has mainly focused on the implementation and improvement of conductive pastes based on fillers, such as silver nanoparticles or carbon-based materials, as part of low-cost solar cells, organic light emitting diodes and wearable electrochemical devices. These inks are mostly based on synthetic polymers and ceramics processed using organic solvents that hamper the addition of active fillers (e.g., chromophores, biological molecules like enzymes and antibodies) to implement sensing devices in a single step that can be pervasively transferred onto multiple surfaces.

Piezoelectric-driven inkjet printing, on the other hand, does not utilize masking equipment and is generally tailored to achieve smaller resolutions and printed areas. Specifically, it allows direct transfer of features with sizes in the order of tens of micrometers through drop-by-drop delivery of functionalized inks. Advantages of inkjet printing include low material consumption, versatility, precise droplet-size deposition, and compatibility in both ink composition and typology of a substrate. However, the restricted size (i.e., depending on the nozzle) of the active molecule to be transferred, the limited rheology (i.e., viscosity) of the material to be printed, and the eventual high volatility of the solvents used, are the main drawbacks that hamper a widespread diffusion of this printing technique.

Currently, there remains a need for the development of improved biocompatible ink compositions for use in screen printing techniques that address the aforementioned drawbacks.

### SUMMARY

The present disclosure provides, among other things, biologically-based ink compositions for printing applications, methods of making the biocompatible ink compositions, as well as articles, objects, devices, and apparatuses that comprise or are manufactured from comprise the biocompatible ink composition.

Some aspects of the present disclosure provide a biologically-based ink composition that comprises a silk fibroin solution having a concentration of silk fibroin in a range of 0.1 wt % to at least about 10 wt %. The biologically-based ink composition further includes a thickening agent and a humectant dispersed throughout the composition, where the thickening agent comprises a polysaccharide.

In further aspects, the thickening agent and the humectant are present in the composition in a weight ratio of between 4:1 and 1:4 or between 2:1 and 1:2.

In some aspects, the concentration of thickening agent in the composition is between 0.1 wt % and 10 wt %, or is between 0.1 wt % and 4 wt %.

In further aspects, the concentration of humectant in the composition is between 0.1 wt % and 10 wt %.

In some aspects, the viscosity of the biologically-based ink composition is between 1,000 cP and 10,000 cP measured at room temperature, or is between 3,000 cP and 5,000 cP measured at room temperature. In some aspects, the biologically-based ink composition has a viscosity suitable for use in screen printing. In further aspects, the biologically-based ink composition has a viscosity that is between 1,000 cP and 2,500 cP when extruded or printed at a shear rate of 100 to 200 s⁻¹.

In some aspects, the polysaccharide is selected from the group consisting of an algin, an alginate, a guaran, a chitosan, a cellulose, and an arabinoxylan. In further aspects, the polysaccharide is sodium alginate. In some aspects, the humectant is glycerol.

In further aspects, the silk fibroin solution is an aqueous silk fibroin solution substantially free of organic solvent. In some aspects, the silk fibroin has a molecular weight of about 3.5 kD to about 350 kD.

In further aspects, the silk fibroin solution further comprises one or more additive or dopant. The additive or dopant may comprise a sensing dye or agent, such as a temperature sensitive dye or agent, a pH sensitive dye or agent, a pressure sensitive dye or agent, a light sensitive dye or agent, a potentiometric sensitive dye or agent, a chemi-sensitive dye or agent, and combinations thereof.

In some aspects, the additive or dopant comprises a conductive additive.

In further aspects, the biologically-based ink composition further includes a biologically active agent or therapeutic agent.

In some aspects, the present disclosure provides an apparatus comprising a substrate and a biologically-based ink composition adhered to a surface of the substrate. The biologically-based ink composition comprises a silk fibroin solution having a concentration of silk fibroin in a range of 0.1 wt % to at least about 10 wt %. The composition further includes a thickening agent and a humectant dispersed throughout the composition, where the thickening agent comprises a polysaccharide.

In further aspects, the substrate is characterized by its flexibility, such that when the printed article contacts an object it substantially conforms to the object's surface. The substrate may comprise a textile, such as cotton, paper, or silk.

In some aspects, the substrate is characterized as having pores, and wherein the biologically-based ink composition is adhered to at least a fraction of the pores. In further aspects, the substrate is characterized as a rigid object.

In some aspects, the biologically-based ink composition forms a pattern on the surface of the substrate. The biologically-based ink composition may include a sensing dye or agent and the pattern on the substrate may take the form of a sensor or environmentally-sensitive sensor. In further aspects, the pattern may span across the entire surface of the substrate. In some aspects, the pattern comprises one or more microchannel.

### Definitions

In this application, unless otherwise clear from context, (i) the term “a” may be understood to mean “at least one”; (ii) the term “or” may be understood to mean “and/or”; (iii) the terms “comprising” and “including” may be understood to encompass itemized components or steps whether presented by themselves or together with one or more additional components or steps; and (iv) the terms “about” and “approximately” are used as equivalents and may be understood to permit standard variation as would be understood by those of ordinary skill in the art; and (v) where ranges are provided, endpoints are included.

Agent: The term “agent” as used herein may refer to a compound or entity of any chemical class including, for example, polypeptides, nucleic acids, saccharides, lipids, small molecules, metals, or combinations thereof. As will be clear from context, in some aspects, an agent can be or comprise a cell or organism, or a fraction, extract, or component thereof. In some aspects, an agent is or comprises a natural product in that it is found in and/or is obtained from nature. In some aspects, an agent is or comprises one or more entities that is man-made in that it is designed, engineered, and/or produced through action of the hand of man and/or is not found in nature. In some aspects, an agent may be utilized in isolated or pure form; in some aspects, an agent may be utilized in crude form. In some aspects, potential agents are provided as collections or libraries, for example that may be screened to identify or characterize active agents within them. Some particular aspects of agents that may be utilized in accordance with the present disclosure include small molecules, antibodies, antibody fragments, aptamers, nucleic acids (e.g., siRNAs, shRNAs, DNA/RNA hybrids, antisense oligonucleotides, ribozymes), peptides, peptide mimetics, etc. In some aspects, an agent is or comprises a polymer. In some aspects, an agent is not a polymer and/or is substantially free of any polymer. In some aspects, an agent contains at least one polymeric moiety. In some aspects, an agent lacks or is substantially free of any polymeric moiety.

Approximately: As used herein, the term “approximately” or “about,” as applied to one or more values of interest, refers to a value that is similar to a stated reference value. In certain aspects, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: Two events or entities are “associated” with one another, as that term is used herein, if the presence, level and/or form of one is correlated with that of the other. For example, a particular entity (e.g., polypeptide, genetic signature, metabolite, etc) is considered to be associated with a particular disease, disorder, or condition, if its presence, level and/or form correlates with incidence of and/or susceptibility to the disease, disorder, or condition (e.g., across a relevant population). In some aspects, two or more entities are physically “associated” with one another if they interact, directly or indirectly, so that they are and/or remain in physical proximity with one another. In some aspects, two or more entities that are physically associated with one another are covalently linked to one another; in some aspects, two or more entities that are physically associated with one another are not covalently linked to one another but are non-covalently associated, for example by means of hydrogen bonds, van der Waals interaction, hydrophobic interactions, magnetism, and combinations thereof.

Biocompatible: The term “biocompatible”, as used herein, may refer to materials that do not cause significant harm to living tissue when placed in contact with such tissue, e.g., in vivo. In certain aspects, materials are “biocompatible” if they are not toxic to cells. In certain aspects, materials are “biocompatible” if their addition to cells in vitro results in less than or equal to 20% cell death, and/or their administration in vivo does not induce significant inflammation or other such adverse effects.

Biodegradable: As used herein, the term “biodegradable” may refer to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain aspects, components generated by breakdown of a biodegradable material are biocompatible and therefore do not induce significant inflammation and/or other adverse effects in vivo. In some aspects, biodegradable polymer materials break down into their component monomers. In some aspects, breakdown of biodegradable materials (including, for example, biodegradable polymer materials) involves hydrolysis of ester bonds. Alternatively or additionally, in some aspects, breakdown of biodegradable materials (including, for example, biodegradable polymer materials) involves cleavage of urethane linkages. Exemplary biodegradable polymers include, for example, polymers of hydroxy acids such as lactic acid and glycolic acid, including but not limited to poly(hydroxyl acids), poly(lactic acid)(PLA), poly(glycolic acid)(PGA), poly(lactic-co-glycolic acid)(PLGA), and copolymers with PEG, polyanhydrides, poly(ortho)esters, polyesters, polyurethanes, poly(butyric acid), poly(valeric acid), poly(caprolactone), poly(hydroxyalkanoates, poly(lactide-co-caprolactone), blends and copolymers thereof. Many naturally occurring polymers are also biodegradable, including, for example, proteins such as albumin, collagen, gelatin and prolamines, for example, zein, and polysaccharides such as alginate, cellulose derivatives and polyhydroxyalkanoates, for example, polyhydroxybutyrate blends and copolymers thereof. Those of ordinary skill in the art will appreciate or be able to determine when such polymers are biocompatible and/or biodegradable derivatives thereof (e.g., related to a parent polymer by substantially identical structure that differs only in substitution or addition of particular chemical groups as is known in the art).

Composition: A “composition” or “ink composition” according to this disclosure may refer to the combination of two or more agents as provided herein to form a composition which is applied to a substrate. The ink composition may contain a molecule which adds a functionality, such responding to environmental stimuli. In this case, the ink composition may be considered a “ink composition”.

Green Chemistry: As used herein, “green chemistry” refers to methods of producing a composition or compound using methods which may lessen harm on the environment and the reagents using these methods may be non-toxic. In some aspects, methods using green chemistry may reduce the consumption of nonrenewable resources.

“Improve,” “increase” or “reduce:” as used herein or grammatical equivalents thereof, indicate values that are relative to a baseline measurement, such as a measurement in the same individual prior to initiation of a treatment provided herein, or a measurement in a control individual (or multiple control individuals) in the absence of the treatment provided herein. In some aspects, a “control individual” is an individual afflicted with the same form of disease or injury as an individual being treated.

Subject: By “subject” is meant a mammal (e.g., a human). In some aspects, a subject is suffering from a relevant disease, disorder or condition. In some aspects, a subject is susceptible to a disease, disorder, or condition. In some aspects, a subject displays one or more symptoms or characteristics of a disease, disorder or condition. In some aspects, a subject does not display any symptom or characteristic of a disease, disorder, or condition. In some aspects, a subject is someone with one or more features characteristic of susceptibility to or risk of a disease, disorder, or condition. In some aspects, a subject is a patient. In some aspects, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena

Therapeutic agent: As used herein, the phrase “therapeutic agent” in general refers to any agent that elicits a desired pharmacological effect when administered to an organism. In some aspects, an agent is considered to be a therapeutic agent if it demonstrates a statistically significant effect across an appropriate population. In some aspects, the appropriate population may be a population of model organisms. In some aspects, an appropriate population may be defined by various criteria, such as a certain age group, gender, genetic background, preexisting clinical conditions, etc. In some aspects, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some aspects, a “therapeutic agent” is an agent that has been or is required to be approved by a government agency before it can be marketed for administration to humans. In some aspects, a “therapeutic agent” is an agent for which a medical prescription is required for administration to humans.

### BRIEF DESCRIPTION OF THE DRAWINGS

FIG. 1 is a biologically-based ink composition comprising a conductive additive printed on a soft functional substrate. The biologically-based ink composition is printed on a centimeter-scale and includes technical apparel having conductive traces consisting of a non-toxic conductive ink. Inks and patterns formed using a green chemistry approach and are integrated into technical apparel and fabric to form functionalized wearable devices.

FIG. 2 shows an example of a decimeter-scale soft functional object including lightweight furniture which has been printed using directional tensile screen-printed fabric for lounge furniture. Ink compositions are able to sense contaminants such as bacteria, or toxic spills.

FIG. 3 shows meter-scale soft functional wall paper which has been screen-printed with silk-based ink compositions using CMYK (cyan, magenta, yellow, and key (black)) color separation. In one example, silk-based ink compositions forming wall art is functionalized with molecules able to sense air quality.

FIG. 4 shows a local demonstration of color change in response to the presence of contamination. Panel (A) shows an ink composition sensitive to contamination. Panel (B) shows the ink composition changing color in response to sensing bacterial contamination.

FIG. 5 shows examples of functional inks printed on different surfaces. Panel (A) shows antibiotic inks printed on a bacterial lawn. Panel (B) shows enzyme inks on paper. Panel (C) shows bone deposition in predetermined geometries on a petri dish based on a pattern of ink containing growth factor such as BMP-8 (bone morphogenic protein) on the petri dish. Panel (D) shows inks printed on a nitrile lab glove that initially are blue inks and change color to red as shown in Panel (E) when the nitrile glove is exposed to bacteria.

FIG. 6 shows the steps used to transfer a conceptual design into a functional screen-printing pattern, including forming a design on a screen and transferring the design onto a substrate to create a paper-based final product. Drawings are used to optimize printing parameters such as patterns, dimensions, resolution and screen formats as shown in panel (A) and panel (B). Patterns are transferred on to the screen as shown panel (C), panel (D), panel (E), and panel (F). The materials for transferring the ink composition to a screen are shown in panel (G), applying the ink composition to a screen is shown in panel (H), and using a squeegee to apply the ink composition to a substrate in shown panel (I). The printed substrate is removed from the screen and allowed to dry as shown in panel (J).

FIG. 7 shows pH-sensing designs tested on silk fabric substrates with ink compositions. Panel (A), panel (B), panel (C), panel (D), panel (E), and panel (F) show examples of pH sensing fabrics that can be implemented using screen printing.

FIG. 8 panels (A)-(D) show pressure-based extrusion fabrication of thickened active ink compositions for temperature sensing objects.

### DETAILED DESCRIPTION

In some aspects, the present disclosure provides biologically-based ink compositions, methods of making the biologically-based ink composition, as well as articles, objects, devices and/or apparatuses fabricated from or that comprise the biologically-based ink compositions. In some aspects, the biologically-based ink compositions are suitable for printing, applying, coating, extending, and/or extruding on a substrate. Suitable substrates include, but are not limited to, soft and conformal substrates (e.g., paper, textiles), as well as hard substrates (e.g., plastic, ceramic, metal). Various aspects according to the present disclosure are described in detail herein.

In some aspects, the present disclosure utilizes the biologically-based ink compositions to functionalize a substrate for use in the fabrication of low-cost, environmentally-sensitive surfaces that directly sense, monitor, and interact with the local or global environment. The environmentally-sensitive surfaces may actively respond to stimulus for distributed monitoring, or may be configured to acquire one or more parameter indicative of the local or global environment (e.g., pH, concentration of compound, conductivity), which may be relayed to additional devices, such as a computing device, for further processing. The biologically-based ink compositions may be patterned on a substrate to form sensors, non-toxic conductive inks, microfluidic channels (e.g., for diagnostics), technical apparel or fashion accessories (e.g., clothes or accessories that monitor environmental and physiological signals), lightweight furniture, tensile canopies, architectural wall paper, façade components, or may be patterned on a substrate to encapsulate scents, flavors, dyes and pigments, therapeutic agents, or biologically active molecules that, in some aspects, may be encapsulated without troublesome use of hydration or refrigeration.

In addition applying, extending, extruding, and/or printing ink compositions, the present disclosure encompasses a recognition that prior limits on a size of a substrate or an area to be printed, do not constrain biologically-based ink compositions of the present disclosure to functionalize large format substrates like textiles.

Indeed, implementations of the present disclosure are useful for a wide range of applications, including but not limited to: biomedical and tissue engineering, consumer products, drug delivery, imaging, medical/surgical devices, optoelectronics, photonics, sensors, synthetic biology, and/or therapeutics.

In some aspects, the biologically-based ink compositions comprise a biopolymer and a solvent or dispersing medium. In some aspects, the biologically-based ink composition may include one or more additional compound or species, such as a thickening agent, a humectant, a plasticizer, a viscosity-modifying agent, and an additive, dopant, or biologically active agent.

In some aspects, the solvent in the biologically-based ink composition comprises an aqueous solution including, but not limited to, water, cell culture medium, buffers (e.g., phosphate buffered saline, buffered solutions (e.g., PBS), Dulbecco's Modified Eagle Medium, fetal bovine serum, or suitable combinations and/or mixtures thereof. In some aspects, the solvent is substantially free of organic solvents (e.g., the biologically-based ink composition contains no detectable organic solvent or contains organic solvent at a level that one of ordinary skill in the pertinent art would consider negligible for a particular use, such as for affecting biocompatibility). In some aspects, the solvent comprises an organic solvent, such as hexafluoroisopropanol.

In some aspects, the biopolymer includes a polypeptide, fragment or variant thereof. Suitable polypeptides include, for example, actins, collagens, catenins, claudins, coilins, elastins, elaunins, extensins, fibrillins, fibroins, keratins, lamins, laminins, silks, structural proteins, tublins, zein proteins, derivatives and combinations thereof.

In some aspects, the biopolymer comprises silk fibroin. As used herein, “silk fibroin” or “SF”, may refer to a biopolymer produced from silkworm fibroin and insect or spider silk protein. See e.g., Lucas et al., 13 Adv. Protein. For example, silk fibroin useful for the present disclosure may be that produced by a number of species, including, without limitation: Antheraea mylitta; Antheraea pernyi; Antheraea yamamai; Galleria mellonella; Bombyx mori; Bombyx mandarina; Galleria mellonella; Nephila clavipes; Nephila senegalensis; Gasteracantha mammosa; Argiope aurantia; Araneus diadematus; Latrodectus geometricus; Araneus bicentenarius; Tetragnatha versicolor; Araneus ventricosus; Dolomedes tenebrosus; Euagrus chisoseus; Plectreurys tristis; Argiope trifasciata; and Nephila madagascariensis. Alternatively, silk utilized in the present disclosure may be prepared through an artificial process, for example, involving genetic engineering of cells or organisms (e.g., genetically engineered bacteria, yeast, mammalian cells, non-human organisms, including animals, or transgenic plants).

Even among biopolymers, silk fibroin is a fascinating material, extensively investigated for its potential in textile, biomedical, photonic and electronic applications. SF is a structural protein, like collagen, but with a unique feature: it is produced from the extrusion of an amino-acidic solution by a living complex organism (while collagen is produced in the extracellular space by self-assembly of cell-produced monomers). SF properties are derived from its structure, which consists of hydrophobic blocks staggered by hydrophilic, acidic spacers. In its natural state, SF is organized in β-sheet crystals alternated with amorphous regions, which provide strength and resilience to the protein. The multiplicities of forms in which regenerated SF can be process at a high protein concentration and molecular weight make it attractive for several high-tech applications.

The degree of crystallinity of the protein can be finely tuned and it influences SF's biological, physical, biochemical and mechanical properties. In addition, the amino-acidic nature of SF brings a diversity of side chain chemistries that allows for the incorporation and stabilization of macromolecules useful in drug delivery applications or in providing cellular instructions. In particular, we have recently showed that dry SF with diverse degrees of crystallinity stabilizes vaccines and antibiotics, eliminating the need for the cold chain. SF is indeed considered a platform technology in biomaterials fabrication as its robustness and qualities bring the assets to add a large portfolio of distinct features (e.g. nanopatterning, biochemical functionalization) to the final construct. Processing of regenerated SF generally involves the partial or total dehydration of a fibroin solution (protein content of 1-15 wt %) to form films, sponges, gels, spheres (micron- to nano-sized) and foams with numerous techniques (e.g. solvent casting, freeze drying, salt leaching, sonication). The rationale beyond these fabrication processes is to manufacture a robust material that combines mechanical strength with biochemical properties.

In some aspects, the biologically-based ink compositions may be formulated from a silk fibroin solution. The silk fibroin solutions used in methods and compositions provided herein may be obtained from a solution containing a dissolved silkworm silk, such as, for example, from Bombyx mori. Alternatively, the silk fibroin solution may be obtained from a solution containing a dissolved spider silk, such as, for example, from Nephila clavipes. The silk fibroin solution can also be obtained from a solution containing a genetically engineered silk such as from bacteria, yeast, mammalian cells, transgenic animals or transgenic plants. See, for example, WO 97/08315 and U.S. Pat. No. 5,245,012. In some aspects, genetically engineered silk can, for example, comprise a therapeutic agent, e.g., a fusion protein with a cytokine, an enzyme, or any number of hormones or peptide-based drugs, antimicrobials and related substrates.

In some aspects, silk fibroin solution can be prepared by any conventional method known to one skilled in the art. In some aspects, a silk solution is an aqueous silk solution.

Silkworm cocoon silk contains two structural proteins, the fibroin heavy chain (˜350 kDa); and the fibroin light chain (˜25 kDa), which are associated with a family of non-structural proteins termed sericins, that glue the fibroin chains together in forming the cocoon. The heavy and light fibroin chains are linked by a disulfide bond at the C-terminus of the two subunits (see Takei, et al. J. Cell Biol., 105: 175, 1987; see also Tanaka, et al J. Biochem. 114: 1, 1993; Tanaka, et al Biochim. Biophys. Acta., 1432: 92, 1999; Kikuchi, et al Gene, 110: 151, 1992). The sericins are a high molecular weight, soluble glycoprotein constituent of silk which gives the stickiness to the material. These glycoproteins are hydrophilic and can be easily removed from cocoons by boiling in water. This process is often referred to as “degumming.”

In some aspects, silk polypeptide compositions utilized in accordance with the present invention are substantially free of sericins (e.g., contain no detectable sericin or contain sericin at a level that one of ordinary skill in the pertinent art will consider negligible for a particular use).

For example, B. mori cocoons are boiled for about 30 minutes in an aqueous solution. In some aspects, the aqueous solution is about 0.02M Na₂CO₃. In some aspects, boiling (degumming) time is in a range of about 5 minutes to about 120 minutes. In some aspects, boiling (degumming) temperature is in a range of about 30° C. to about 120° C. The cocoons may be rinsed, for example, with water to extract the sericin proteins and the extracted silk is dissolved in an aqueous salt solution. Exemplary salts useful for this purpose include lithium bromide, lithium thiocyanate, calcium nitrate or other chemicals capable of solubilizing silk. Preferably, in some aspects, the extracted silk is dissolved in about 9-12 M LiBr solution. The salt is consequently removed using, for example, dialysis.

If necessary, the solution can then be concentrated using, for example, dialysis against a hygroscopic polymer, for example, PEG, a polyethylene oxide, amylose or sericin. In some aspects, PEG has a molecular weight of 8,000-10,000 g/mol and has a concentration of 25-50%. A slide-a-lyzer dialysis cassette (Pierce, MW CO 3500) is preferably used. However, any dialysis system can be used. The dialysis is for a time period sufficient to result in a final concentration of aqueous silk solution between 10-30%. In most cases dialysis for 2-12 hours is sufficient.

It should be noted that certain structural proteins, including silk fibroin, exhibit an inherent self-assembly property. In some aspects, this process involves the formation of beta-sheet secondary structure within a structural protein (or fragments). In some aspects, the biopolymer in the biologically-based inks contains a range of degrees/levels of beta-sheet crystallinity. For example, the biopolymer may contain a beta-sheet content ranging between about 5% and 70%, e.g., about 5%, about 10%, about 15%, about 20%, about 25%, about 30%, about 35%, about 40%, about 45%, about 50%, about 55%, about 60%, about 65% or about 75%.

The β-sheet conformation improves the interaction between the silk contained in the biologically-based ink compositions and the diverse functionalized substrates. Depending on the type of printing, the molecular weight of the silk employed to realize the ink compositions needs to be tuned accordingly. This is achieved during the degumming step lasting between 30 minutes (e.g., for extrusion and screen printing compounds) up to two hours (e.g. for ink-jet printing).

In some aspects, the biopolymers in the biologically-based ink are below 200 kDa, or below 150 kDa. In some aspects, the biopolymers (or fragments thereof) in the biologically-based ink compositions have a molecular weight ranging between about 3.5 kDa and about 120 kDa. For example, the biologically-based ink compositions may have a molecular weight ranging between about 3.5-110 kDa, about 3.5-100 kDa, about 3.5-90 kDa, about 3.5-80 kDa, about 3.5-70 kDa, about 3.5-60 kDa, about 3.5-50 kDa, about 3.5-40 kDa, about 3.5-35 kDa, about 3.5-30 kDa, about 3.5-25 kDa, about 3.5-20 kDa, about 50-120 kDa, about 60-120 kDa, about 70-120 kDa, about 80-120 kDa, or about 90-120 kDa. In some aspects, such structural protein may be a full-length (e.g., wild type) structural protein having a molecular weight falling within any of the ranges shown above. In other aspects, such structural protein may be so-called “low molecular weight protein,” i.e., corresponding to reduced size fragments of a full-length counterpart, for example fragments of the full-length counterpart.

In some aspects, no more than 15% of the total number of silk fibroin fragments in biologically-based ink composition have a molecular weight exceeding 200 kDa, and at least 50% of the total number of the silk fibroin fragments in the population has a molecular weight within a specified range, wherein the specified range is between about 3.5 kDa and about 120 kDa. Low molecular weight silk fibroin is described in detail in U.S. provisional application 61/883,732, entitled “LOW MOLECULAR WEIGHT SILK FIBROIN AND USES THEREOF,” the entire contents of which are incorporated herein by reference.

In some aspects, the consistency of the biologically-based ink composition may be further enhanced by selectively enriching certain range or ranges of fragment size (molecular weight) in a preparation. In some aspects, therefore, a step of filtration may be included during the preparation of such an ink composition. For instance, filters with a known cut-off range (such as 0.2 μm) may be used to remove any fragments or aggregates (e.g., contamination) that are larger than the pore size.

Alternatively or additionally, in some aspects, protein solution may be further processed, including extended heating and/or high pressure treatment, in order to promote fragmentation of large structural proteins.

In some aspects, the biologically-based ink composition may be heated (such as by boiling at atmospheric pressure) during the process of biopolymer preparation for a period of time, e.g., for about 10 minutes, about 15 minutes, about 20 minutes, about 30 minutes, about 40 minutes, about 45 minutes, about 50 minutes, about 60 minutes, about 70 minutes, about 80 minutes, about 90 minutes, about 100 minutes, about 110 minutes, about 120 minutes, or longer.

Alternatively or additionally, the biopolymer solution may be heated or boiled at an elevated temperature. For example, in some aspects, the biopolymer solution can be heated or boiled at about 101.0° C., at about 101.5° C., at about 102.0° C., at about 102.5° C., at about 103.0° C., at about 103.5° C., at about 104.0° C., at about 104.5° C., at about 105.0° C., at about 105.5° C., at about 106.0° C., at about 106.5° C., at about 107.0° C., at about 107.5° C., at about 108.0° C., at about 108.5° C., at about 109.0° C., at about 109.5° C., at about 110.0° C., at about 110.5° C., at about 111.0° C., at about 111.5° C., at about 112.0° C., at about 112.5° C., at about 113.0° C., 113.5° C., at about 114.0° C., at about 114.5° C., at about 115.0° C., at about 115.5° C., at about 116.0° C., at about 116.5° C., at about 117.0° C., at about 117.5° C., at about 118.0° C., at about 118.5° C., at about 119.0° C., at about 119.5° C., at about 120.0° C., or higher.

In some aspects, such elevated temperature can be achieved by carrying out at least portion of the heating process (e.g., boiling process) under pressure. For example, suitable pressure under which protein fragments provided herein can be produced are typically between about 10-40 psi, e.g., about 11 psi, about 12 psi, about 13 psi, about 14 psi, about 15 psi, about 16 psi, about 17 psi, about 18 psi, about 19 psi, about 20 psi, about 21 psi, about 22 psi, about 23 psi, about 24 psi, about 25 psi, about 26 psi, about 27 psi, about 28 psi, about 29 psi, about 30 psi, about 31 psi, about 32 psi, about 33 psi, about 34 psi, about 35 psi, about 36 psi, about 37 psi, about 38 psi, about 39 psi, or about 40 psi.

Alternatively or additionally, the biopolymer solution may be further processed, including centrifugation.

In some aspects, the biologically-based inks have low dissolved gas contents. In some aspects, a step of degassing may be optionally performed prior to printing in order to enhance printing quality.

In some particular aspects, ink compositions for use in accordance with the present disclosure include a biopolymer, for example, silk whose molecular weight is within a range of about 1 kDa to about 400 kDa. In some aspects, a molecular weight is about 1 kDa, about 5 kDa, about 10 kDa, about 15 kDa, about 20 kDa, about 25 kDa, about 30 kDa, about 40 kDa, about 50 kDa, about 60 kDa, about 70 kDa, about 80 kDa, about 90 kDa, about 100 kDa, about 150 kDa, about 200 kDa, about 250 kDa, about 300 kDa, about 350 kDa, and/or about 400 kDa.

While silk fibroin extraction methods generally have been well documented, the present disclosure encompasses the recognition that certain structural proteins can be processed further to be made suitable for bio-printing provided herein, thereby overcoming previously existed hurdles that had prevented the use of certain structural proteins for printing purposes.

Accordingly, in some aspects, such methods involve extraction of structural proteins (such as silk fibroin) under high temperature, such as between about 101-135° C., between about 105-130° C., between about 110-130° C., between about 115-125° C., between about 118-123° C., e.g., about 115° C., 116° C., 117° C., 118° C., 119° C., 120° C., 121° C., 122° C., 123° C., 124° C., 125° C.

Additionally or alternatively, provided methods in some aspects involve extraction of structural proteins (such as silk fibroin) under elevated pressure, such as about 5 psi, 6 psi, 7 psi, 8 psi, 9 psi, 10 psi, 11 psi, 12 psi, 13 psi, 14 psi, 15 psi, 16 psi, 17 psi, 18 psi, 19 psi, 20 psi, 21 psi, 22 psi, 23 psi, 24 psi, 25 psi, 30 psi, 31 psi, 32 psi, 33 psi, 34 psi and 35 psi. In some aspects, structural proteins (such as silk fibroin) are extracted under high temperature and under elevated pressure, e.g., at about 110-130° C. and about 10-20 psi for a duration suitable to produce a protein solution that would easily go through a 0.2 μm filter. In some aspects, structural proteins (such as silk fibroin) are extracted under high temperature and under elevated pressure, e.g., at about 110-130° C. and about 10-20 psi for about 60-180 minutes. In some aspects, structural proteins (such as silk fibroin) are extracted under high temperature and under elevated pressure, e.g., at about 116-126° C. and about 12-20 psi for about 90-150 minutes.

In accordance with various aspects, a silk solution may comprise any of a variety of concentrations of silk fibroin. In some aspects, a silk solution may comprise 0.1 to 40% by weight silk fibroin. In some aspects, a silk solution may comprise between about 0.5% and 40% (e.g., 0.5% to 25%, 0.5% to 20%, 0.5% to 15%, 0.5% to 10%, 0.5% to 5%, 0.5% to 1.0%) by weight silk fibroin, inclusive. In some aspects, a silk solution may comprise at least 0.1% (e.g., at least 0.5%, 1%, 2%, 3%, 4%, 5%, 6%, 7%, 8%, 9%, 10%, 15%, 20%, 25%, 30%, 35%, 40%) by weight silk fibroin. In some aspects, a silk solution may comprise at most 40% (e.g., at most 40%, 35%, 30%, 25%, 20%, 15%, 14%, 13%, 12% 11%, 10%, 5%, 4%, 3%, 2%, 1%) by weight silk fibroin.

In some aspects provided herein, compositions and methods are particularly amenable to the incorporation of labile molecules, such as bioactive agents or therapeutics, and can, in certain aspects, be used to produce controlled release biomaterials. In some aspects, methods are performed in water only. In some aspects, methods are performed in an absence of organic solvents.

Alternatively, in some aspects, the silk fibroin solution can be produced using organic solvents, for example hexafluoroisopropanol (HFIP). Such methods have been described, for example, in Li, M., et al., J. Appi. Poly Sci. 2001, 79, 2192-2199; Mm, s., et al. Sen I Gakkaishi 1997, 54, 85-92; Nazarov, R. et al., Biomacromolecules 2004 May-June; 5(3):71 8-26.

In some aspects, silk fibroin solutions used to prepare silk ink compositions are substantially free of sericin (i.e., degummed). In some aspects, silk fibroin useful for the preparation of silk ink compositions described herein are extracted from cocoons (i.e., natural source of silk fibers). In some aspects, silk fibroin useful for the preparation of silk ink compositions described herein are recombinantly produced. In some aspects, silk fibroin useful for the preparation of silk ink compositions described herein are low molecular weight silk fibroin.

In some aspects, the biologically-based ink compositions comprise a thickening agent. The thickening agent may be used to adjust the viscosity of the biologically-based ink composition, for example, to formulate the composition to a range that is suitable for a particular printing method. Suitable thickening agents include polysaccharides or ionic polysaccharides including, but not limited to, algin, alginates (e.g., algin salts, such as sodium alginate, calcium alginate, or potassium alginate), guar gum (e.g., guaran), chitosan, cellulose, arabinoxylan, derivatives and mixtures thereof. In some aspects, the biologically-based ink composition may contain between about 0.1 wt % to 20 wt % of the thickening agent, e.g., 0.5 wt %, 0.6 wt %, 0.7 wt %, 0.8 wt %, 0.9 wt %, 1 wt %, 1.1 wt %, 1.2 wt %, 1.3 wt %, 1.4 wt %, 1.5 wt %, 1.6 wt %, 1.7 wt %, 1.8 wt %, 1.9 wt %, 2 wt %, 3, wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, or 20 wt %.

In some aspects, the thickening agent is present in the biologically-based ink composition in an amount sufficient to cause the viscosity of the ink composition to range between 10 poise to 100 poise. In some aspects, the thickening agent is present in the biologically-based ink composition to cause the viscosity of the ink composition to be greater than 10 poise, 15 poise, 20 poise, 30 poise, 40 poise, 50 poise, 60 poise, 70 poise, 80 poise, 90 or greater. In some aspects, the thickening agent is present in the biologically-based in composition to cause the viscosity of the ink composition to be about 10 poise, 15 poise, 20 poise, 30 poise, 40 poise, 50 poise, 60 poise, 70 poise, 80 poise, 90 poise, or about 100 poise. In some aspects, the biologically-based ink compositions having viscosities in the range of 10 poise to 100 poise may be suitable for use in screen-printing applications. Viscosities suitable for use in inkjet printing are typically much lower (e.g., in the range of 1 cP to 20 cP), as these viscosities are less prone to clog the nozzles during inkjet deposition.

In some aspects, the thickening agent is inert in the biologically-based ink composition. As used herein, “inert” may refer to a compound or species that does not react with other components of the ink composition, or may refer to compounds or species that undergo negligible reactions with other components in the ink composition.

In some aspects, polysaccharides such as sodium alginate or guar gum are advantageous for various aspects disclosed herein as these thickening agents may be added to the ink composition without affecting any performance (e.g., the resolution and scale of the printed object, and undergo negligible reactions, if any, with other components of the ink composition) and may be washed off after transfer via screen printing. In addition, according to various aspects, doping the biologically-based ink compositions in a sufficient amount of polysaccharides achieves surprising and unexpected rheological properties that allow the biologically-based ink compositions to be screen printed onto substrates over larger areas when compared to conventional inks and techniques. That is, previous bio-inks have typically been optimized for inkjet printing whose active areas are small in scale. Demonstrated herein, the biologically-based ink compositions may be printed (e.g., using screen-printing) onto substrates, such as textiles, in a highly reproducible fashion to achieve unprecedented printing areas (e.g., on the order of square meters), while achieving high resolutions (e.g., down to hundreds of μm) with one or more additive embedded in the ink to achieve, for example, multi spectral combinations of colors on the substrate. In some aspects, the biologically-based ink compositions may be screen-printed and reproducibly scaled-up for mass production.

In some aspects, one or more additional viscosity modifying agent may be present in the biologically-based ink composition. Suitable viscosity modifying agents include viscosity modifiers that may be included in the inks include, but are not limited to: acrylate esters, acrylic esters, acrylic monomer, aliphatic mono acrylate, aliphatic mono methacrylate, alkoxylated lauryl acrylate, alkoxylated phenol acrylate, alkoxylated tetrahydrofurfuryl acrylate, C12-C14 alkyl methacrylate, aromatic acrylate monomer, aromatic methacrylate monomer, caprolactone acrylate, cyclic trimethylol-propane formal acrylate, cycloaliphatic acrylate monomer, dicyclopentadienyl methacrylate, diethylene glycol methylether methacrylate, epoxidized soybean fatty acid esters, epoxidized linseed fatty acid esters, epoxy acrylate, epoxy (meth)acrylate, 2-(2-ethoxy-ethoxy) ethyl acrylate, ethoxylated (4) nonylphenol acrylate, ethoxylated (4) nonyl phenol methacrylate, ethoxylated nonyl phenol acrylate, glucose, fructose, corn syrup, gum syrup, hydroxy-terminated epoxidized 1,3-polybutadiene, isobornyl acrylate, isobornyl methacrylate, isodecyl acrylate, isodecyl methacrylate, isooctyl acrylate, isooctyl methacrylate, lauryl acrylate, lauryl methacrylate, methoxy polyethylene glycol (350) monoacrylate, methoxy polyethylene glycol (350) monomethacrylate, methoxy poly-ethylene glycol (550) monoacrylate, methoxy polyethylene glycol (550) mono-methacrylate, nonyl-phenyl polyoxyethylene acrylate, octyldecyl acrylate, 2-phenoxyethyl acrylate, 2-phenoxyethyl methacrylate, polybutadiene polymer, polyester acrylate, polyester methacrylate, polyether acrylate, polyether methacrylate, polysorbates, stearyl acrylate, stearyl methacrylate, syrups, tetrahydrofurfuryl acrylate, tetrahydrofurfuryl methacrylate, triethylene glycol ethyl ether methacrylate, 3,3,5-trimethylcyclohexyl methacrylate, urethane acrylate and urethane methacrylate and combinations thereof.

In some aspects, the biologically-based ink composition comprises a humectant or plasticizer. In general, a humectant is a water soluble solvent and any one of a group of hygroscopic substances with hydrating properties, i.e., used to keep things moist. They often are a molecule with several hydrophilic groups, most often hydroxyl groups; however, amines and carboxyl groups, sometimes esterified, can be encountered as well (its affinity to form hydrogen bonds with molecules of water, is the crucial trait). Humectants may help maintain the flexibility of the final object or of the substrate (e.g. textiles) on which they are transferred, in the case of screen printing. Similarly, plasticizers may be added to the ink composition to produce or promote flexibility and to reduce brittleness. In some aspects, the biologically-based ink composition may contain between about 0.1 wt % to 20 wt % of the humectant or plasticizer, e.g., 0.5 wt %, 0.6 wt %, 0.7 wt %, 0.8 wt %, 0.9 wt %, 1 wt %, 1.1 wt %, 1.2 wt %, 1.3 wt %, 1.4 wt %, 1.5 wt %, 1.6 wt %, 1.7 wt %, 1.8 wt %, 1.9 wt %, 2 wt %, 3, wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, or 20 wt %.

Non-limiting examples of some humectants or plasticizers include, but are not limited to, glycerol, propylene glycol (E1520), hexylene glycol, and butylene glycol; glyceryl triacetate (E1518); vinyl alcohol; neoagarobiose; Sugar alcohols/sugar polyols: glycerol/glycerin, sorbitol (E420), xylitol, maltitol (E965); polymeric polyols (e.g., polydextrose (E1200)); quillaia (E999); urea; aloe vera gel; MP Diol; alpha hydroxy acids (e.g., lactic acid); and, honey. The chemical compound lithium chloride is an excellent (but toxic) humectant, as well. Typically, humectants such as glycerol and ethylene glycol are used in water-based inks to prevent the nozzle from clogging.

In some aspects, the thickening agent and humectant or plasticizer are present in the biologically-based ink composition in a weight ratio of between 4:1 and 1:4, including but not limited to, between 3:1 and 1:3, between 2:1 and 1:2, and other combinations of the upper and lower bounds of these ranges. In some cases, thickening agent and humectant or plasticizer are present in the biologically-based ink composition in a weight ratio of 4:1, 3:1, 2:1, 1:1, 1:2, 1:3, or 1:4. In some aspects, the biologically-based ink composition contains both the thickening agent and humectant or plasticizer in an amount of about 1 wt %, 2 wt %, 3 wt %, 4 wt %, 5 wt %, 6 wt %, 7 wt %, 8 wt %, 9 wt %, or 10 wt %.

In some aspects, provided aqueous biologically-based ink compositions may contain a surfactant agent which works as a wetting and/or penetrating agent. Use of surfactants in water-based ink compositions is, in some aspects, beneficial because even a relatively small amount of a surfactant can significantly modify or affect the surface tension of an aqueous solution (e.g., water or buffers). In some aspects, a surfactant agent is present at concentrations ranging between about 0.05-20 wt %, e.g., between about 0.1-10 wt % (or by volume) of the biologically-based ink composition.

In some aspects, the composition comprises one or more sensing agents, such as a sensing dye. The sensing agents/sensing dyes are environmentally sensitive and produce a measurable response to one or more environmental factors. In some aspects, the environmentally-sensitive agent or dye may be present in the ink composition in an effective amount to alter the ink composition from a first chemical-physical state to a second chemical-physical state in response to an environmental parameter (e.g., a change in pH, light intensity or exposure, temperature, pressure or strain, voltage, physiological parameter of a subject, and/or concentration of chemical species in the surrounding environment) or an externally applied stimulus (e.g., optical interrogation, acoustic interrogation, and/or applied heat). In some cases, the sensing dye is present to provide one optical appearance under one given set of environmental conditions and a second, different optical appearance under a different given set of environmental conditions. Suitable concentrations for the sensing agents described herein can be the concentrations for the colorants and additives described elsewhere herein. A person having ordinary skill in the chemical sensing arts can determine a concentration that is appropriate for use in a sensing application of the inks described herein.

In some aspects, the first and second chemical-physical state may be a physical property of the biologically-based ink composition, such as mechanical property, a chemical property, an acoustical property, an electrical property, a magnetic property, an optical property, a thermal property, a radiological property, or an organoleptic property. Exemplary sensing dyes or agents include, but are not limited to, a pH sensitive agent, a thermal sensitive agent, a pressure or strain sensitive agent, a light sensitive agent, or a potentiometric agent.

Exemplary pH sensitive dyes or agents include, but are not limited to, cresol red, methyl violet, crystal violet, ethyl violet, malachite green, methyl green, 2-(p-dimethylaminophenylazo) pyridine, paramethyl red, metanil yellow, 4-phenylazodiphenylamine, thymol blue, metacresol purple, orange IV, 4-o-Tolylazo-o-toluindine, quinaldine red, 2,4-dinitrophenol, erythrosine disodium salt, benzopurpurine 4B, N,N-dimethyl-p-(m-tolylazo) aniline, p-dimethylaminoazobenene, 4,4′-bis(2-amino-1-naphthylazo)-2,2′-stilbenedisulfonic acid, tetrabromophenolphthalein ethyl ester, bromophenol blue, congo red, methyl orange, ethyl orange, 4-(4-dimethylamino-1-naphylazo)-3-methoxybenesulfonic acid, bromocresol green, resazurin, 4-phenylazo-1-napthylamine, ethyl red 2-([-dimethylaminophenyazo) pyridine, 4-(p-ethoxypehnylazo)-m-phenylene-diamine monohydrochloride, resorcin blue, alizarin red S, methyl red, propyl red, bromocresol purple, chlorophenol red, p-nitrophenol, alizarin 2-(2,4-dinitrophenylazo) 1-napthol-3,6-disulfonic acid, bromothymol blue, 6,8-dinitro-2,4-(1H) quinazolinedione, brilliant yellow, phenol red, neutral red, m-nitrophenol, cresol red, turmeric, metacresol purple, 4,4′-bis(3-amino-1-naphthylazo)-2,2′-stilbenedisulfonic acid, thymol blue, p-naphtholbenzein, phenolphthalein, o-cresolphthalein, ethyl bis(2,4-dimethylphenyl) ethanoate, thymolphthalein, nitrazine yellow, alizarin yellow R, alizarin, p-(2,4-dihydroxyphenylazo) benzenesulfonic acid, 5,5′-indigodisulfonic acid, 2,4,6-trinitrotoluene, 1,3,5-trinitrobenezne, and clayton yellow.

Exemplary light responsive dyes or agents include, but are not limited to, photochromic compounds or agents, such as triarylmethanes, stilbenes, azasilbenes, nitrones, fulgides, spiropyrans, napthopyrans, spiro-oxzines, quinones, derivatives and combinations thereof.

Exemplary potentiometric dyes include, but are not limited to, substituted amiononaphthylehenylpridinium (ANEP) dyes, such as di-4-ANEPPS, di-8-ANEPPS, and N-(4-Sulfobutyl)-4-(6-(4-(Dibutylamino)phenyl)hexatrienyl)Pyridinium (RH237).

Exemplary temperature sensitive dyes or agents include, but are not limited to, thermochromic compounds or agents, such as thermochromic liquid crystals, leuco dyes, fluoran dyes, octadecylphosphonic acid.

Exemplary pressure or strain sensitive dyes or agents include, but are not limited to, spiropyran compounds and agents.

Exemplary chemi-sensitive dyes or agents include, but are not limited to, antibodies such as immunoglobulin G (IgG) which may change color from blue to red in response to bacterial contamination.

In some aspects, the biologically-based ink compositions comprise one or more additive, dopant, or biologically active agent suitable for a desired intended purpose. In some aspects, the additive or dopant may be present in the biologically-based ink composition in an amount effective to impart an optical or organoleptic property to the ink composition. Exemplary additives or dopants that impart optical or organoleptic properties include, but are not limited to, dyes/pigments, flavorants, aroma compounds, granular or fibrous fillers.

Additionally or alternatively, the additive, dopant, or biologically active agent may be present in the biologically-based ink composition in an amount effective to “functionalize” the ink composition to impart a desired mechanical property or added functionality to the ink composition. Exemplary additive, dopants, or biologically active agent that impart the desired mechanical property or added functionality include, but are not limited to: environmentally sensitive/sensing dyes; active biomolecules; conductive or metallic particles; inorganic particles drugs (e.g., antibiotics, small molecules or low molecular weight organic compounds); proteins and fragments or complexes thereof (e.g., enzymes, antigens, antibodies and antigen-binding fragments thereof); cells and fractions thereof (viruses and viral particles; prokaryotic cells such as bacteria; eukaryotic cells such as mammalian cells and plant cells; fungi).

In some aspects, the additive or dopant comprises a flavoring agent or flavorant. Exemplary flavorants include ester flavorants, amino acid flavorants, nucleic acid flavorants, organic acid flavorants, and inorganic acid flavorants, such as, but not limited to, diacetyl, acetylpropionyl, acetoin, isoamyl acetate, benzaldehyde, cinnamaldehyde, ethyl propionate, methyl anthranilate, limonene, ethyl decadienoate, allyl hexanoate, ethyl maltol, ethylvanillin, methyl salicylate, manzanate, glutamic acid salts, glycine salts, guanylic acids salts, inosinic acid salts, acetic acid, ascorbic acid, citric acid, fumaric acid, lactic acid, malic acid, phosphoric acid, tartaric acid, derivatives, and mixtures thereof.

In some aspects, the additive or dopant comprises an aroma compound. Exemplary aroma compounds include ester aroma compounds, terpene aroma compounds, cyclic terpenes, and aromatic aroma compounds, such as, but not limited to, geranyl acetate, methyl formate, methyl acetate, methyl propionate, methyl butyrate, ethyl acetate, ethyl butyrate, isoamyl acetate, pentyl butrate, pentyl pentanoate, octyl acetate, benzyl acetate, methyl anthranilate, myrecene, geraniol, nerol, citral, cironellal, cironellol, linalool, nerolidol, limonene, camphor, menthol, carone, terpineol, alpha-lonone, thujone, eucalyptol, benzaldehyde, eugenol, cinnamaldehyde, ethyl maltol, vanillin, anisole, anethole, estragole, thymol.

In some aspects, the additive or dopant comprises a colorant, such as a dye or pigment. In some aspects, the dye or pigment imparts a color or grayscale to the biologically-based ink composition. The colorant can be different than the sensing agents and/or sensing dyes below. Any organic and/or inorganic pigments and dyes can be included in the inks. Exemplary pigments suitable for use in the present disclosure include International Color Index or C.I. Pigment Black Numbers 1, 7, 1 1 and 31, C.I. Pigment Blue Numbers 15, 15:1, 15:2, 15:3, 15:4, 15:6, 16, 27, 29, 61 and 62, C.I. Pigment Green Numbers 7, 17, 18 and 36, C.I. Pigment Orange Numbers 5, 13, 16, 34 and 36, C.I. Pigment Violet Numbers 3, 19, 23 and 27, C.I. Pigment Red Numbers 3, 17, 22, 23, 48:1, 48:2, 57:1, 81:1, 81:2, 81:3, 81:5, 101, 1 14, 122, 144, 146, 170, 176, 179, 181, 185, 188, 202, 206, 207, 210 and 249, C.I. Pigment Yellow Numbers 1, 2, 3, 12, 13, 14, 17, 42, 65, 73, 74, 75, 83, 30, 93, 109, 1 10, 128, 138, 139, 147, 142, 151, 154 and 180, D&C Red No. 7, D&C Red No. 6 and D&C Red No. 34, carbon black pigment (such as Regal 330, Cabot Corporation), quinacridone pigments (Quinacridone Magenta (228-0122), available from Sun Chemical Corporation, Fort Lee, N.J.), diarylide yellow pigment (such as AAOT Yellow (274-1788) available from Sun Chemical Corporation); and phthalocyanine blue pigment (such as Blue 15:3 (294-1298) available from Sun Chemical Corporation). The classes of dyes suitable for use in present invention can be selected from acid dyes, natural dyes, direct dyes (either cationic or anionic), basic dyes, and reactive dyes. The acid dyes, also regarded as anionic dyes, are soluble in water and mainly insoluble in organic solvents and are selected, from yellow acid dyes, orange acid dyes, red acid dyes, violet acid dyes, blue acid dyes, green acid dyes, and black acid dyes. European Patent 0745651, incorporated herein by reference, describes a number of acid dyes that are suitable for use in the present disclosure. Exemplary yellow acid dyes include Acid Yellow 1 International Color Index or C.I. 10316); Acid Yellow 7 (C.I. 56295); Acid Yellow 17 (C.I. 18965); Acid Yellow 23 (C.I. 19140); Acid Yellow 29 (C.I. 18900); Acid Yellow 36 (C.I. 13065); Acid Yellow 42 (C.I. 22910); Acid Yellow 73 (C.I. 45350); Acid Yellow 99 (C.I. 13908); Acid Yellow 194; and Food Yellow 3 (C.I. 15985). Exemplary orange acid dyes include Acid Orange 1 (C.I. 13090/1); Acid Orange 10 (C.I. 16230); Acid Orange 20 (C.I. 14603); Acid Orange 76 (C.I. 18870); Acid Orange 142; Food Orange 2 (C.I. 15980); and Orange B.

Exemplary red acid dyes include Acid Red 1. (C.I. 18050); Acid Red 4 (C.I. 14710); Acid Red 18 (C.I. 16255), Acid Red 26 (C.I. 16150); Acid Red 2.7 (C.I. as Acid Red 51 (C.I. 45430, available from BASF Corporation, Mt. Olive, N.J.) Acid Red 52 (C.I. 45100); Acid Red 73 (C.I. 27290); Acid Red 87 (C. I. 45380); Acid Red 94 (C.I. 45440) Acid Red 194; and Food Red 1 (C.I. 14700). Exemplary violet acid dyes include Acid Violet 7 (C.I. 18055); and Acid Violet 49 (C.I. 42640). Exemplary blue acid dyes include Acid Blue 1 (C.I. 42045); Acid Blue 9 (C.I. 42090); Acid Blue 22 (C.I. 42755); Acid Blue 74 (C.I. 73015); Acid Blue 93 (C.I. 42780); and Acid Blue 158A (C.I. 15050). Exemplary green acid dyes include Acid Green 1 (C.I. 10028); Acid Green 3 (C.I. 42085); Acid Green 5 (C.I. 42095); Acid Green 26 (C.I. 44025); and Food Green 3 (C.I. 42053). Exemplary black acid dyes include Acid Black 1 (C.I. 20470); Acid Black 194 (Basantol® X80, available from BASF Corporation, an azo/1:2 CR-complex.

Exemplary direct dyes for use in the present disclosure include Direct Blue 86 (C.I. 74180); Direct Blue 199; Direct Black 168; Direct Red 253; and Direct Yellow 107/132 (C.I. Not Assigned).

Exemplary natural dyes for use in the present disclosure include Alkanet (C.I. 75520, 75530); Annafto (C.I. 75120); Carotene (C.I. 75130); Chestnut; Cochineal (C.I. 75470); Cutch (C.I. 75250, 75260); Divi-Divi; Fustic (C.I. 75240); Hypernic (C.I. 75280); Logwood (C.I. 75200); Osage Orange (C.I. 75660); Paprika; Quercitron (C.I. 75720); Sanrou (C.I. 75100); Sandal Wood (C.I. 75510, 75540, 75550, 75560); Sumac; and Tumeric (C.I. 75300). Exemplary reactive dyes for use in the present disclosure include Reactive Yellow 37 (monoazo dye); Reactive Black 31 (disazo dye); Reactive Blue 77 (phthalo cyanine dye) and Reactive Red 180 and Reactive Red 108 dyes. Suitable also are the colorants described in The Printing Ink Manual (5th ed., Leach et al. eds. (2007), pages 289-299. Other organic and inorganic pigments and dyes and combinations thereof can be used to achieve the colors desired.

In addition to or in place of visible colorants, ink compositions provided herein can contain UV fluorophores that are excited in the UV range and emit light at a higher wavelength (typically 400 nm and above). Examples of UV fluorophores include but are not limited to materials from the coumarin, benzoxazole, rhodamine, napthalimide, perylene, benzanthrones, benzoxanthones or benzothia-xanthones families. The addition of a UV fluorophore (such as an optical brightener for instance) can help maintain maximum visible light transmission. The amount of colorant, when present, generally is between 0.05% to 5% or between 0.1% and 1% based on the weight of the ink composition.

For non-white ink compositions, the amount of pigment/dye generally is present in an amount of from at or about 0.1 wt % to at or about 20 wt % based on the weight of the ink composition. In some applications, a non-white ink can include 15 wt % or less pigment/dye, or 10 wt % or less pigment/dye or 5 wt % pigment/dye, or 1 wt % pigment/dye based on the weight of the ink composition. In some applications, a non-white ink can include 1 wt % to 10 wt %, or 5 wt % to 15 wt %, or 10 wt % to 20 wt % pigment/dye based on the weight of the ink composition. In some applications, a non-white ink can contain an amount of dye/pigment that is 1 wt %, 2 wt %, 3 wt %, 4 wt %, 5%, 6 wt %, 7 wt %, 8 wt %, 9 wt %, 10 wt %, 11 wt %, 12 wt %, 13 wt %, 14 wt %, 15%, 16 wt %, 17 wt %, 18 wt %, 19 wt % or 20 wt % based on the weight of the ink composition.

For white ink compositions, the amount of white pigment generally is present in an amount of from at or about 1 wt % to at or about 60 wt % based on the weight of the ink composition. In some applications, greater than 60 wt % white pigment can be present. Preferred white pigments include titanium dioxide (anatase and rutile), zinc oxide, lithopone (calcined coprecipitate of barium sulfate and zinc sulfide), zinc sulfide, blanc fixe and alumina hydrate and combinations thereof, although any of these can be combined with calcium carbonate. In some applications, a white ink can include 60 wt % or less white pigment, or 55 wt % or less white pigment, or 50 wt % white pigment, or 45 wt % white pigment, or 40 wt % white pigment, or 35 wt % white pigment, or 30 wt % white pigment, or 25 wt % white pigment, or 20 wt % white pigment, or 15 wt % white pigment, or 10 wt % white pigment, based on the weight of the ink composition. In some applications, a white ink can include 5 wt % to 60 wt %, or 5 wt % to 55 wt %, or 10 wt % to 50 wt %, or 10 wt % to 25 wt %, or 25 wt % to 50 wt %, or 5 wt % to 15 wt %, or 40 wt % to 60 wt % white pigment based on the weight of the ink composition. In some applications, a non-white ink can an amount of dye/pigment that is 5%, 6 wt %, 7 wt %, 8 wt %, 9 wt %, 10 wt %, 11 wt %, 12 wt %, 13 wt %, 14 wt %, 15%, 16 wt %, 17 wt %, 18 wt %, 19 wt %, 20 wt %, 21 wt %, 22 wt %, 23 wt %, 24 wt %, 25%, 26 wt %, 27 wt %, 28 wt %, 29 wt %, 30 wt %, 31 wt %, 32 wt %, 33 wt %, 34 wt %, 35%, 36 wt %, 37 wt %, 38 wt %, 39 wt %, 40 wt %, 41 wt %, 42 wt %, 43 wt %, 44 wt %, 45%, 46 wt %, 47 wt %, 48 wt %, 49 wt %, 50 wt %, 51 wt %, 52 wt %, 53 wt %, 54 wt %, 55%, 56 wt %, 57 wt %, 58 wt %, 59 wt % or 60 wt % based on the weight of the ink composition.

In some aspects, the additive or dopant comprises a conductive additive. Exemplary conductive additives include, but are not limited to graphite, graphite powder, carbon nanotubes, and metallic particles or nanoparticles, such as gold nanoparticles. In some aspects, the conductive additive is biocompatible and non-toxic.

In some aspects, the additive is a biologically active agent. The term “biologically active agent” as used herein refers to any molecule which exerts at least one biological effect in vivo. For example, the biologically active agent can be a therapeutic agent to treat or prevent a disease state or condition in a subject. Biologically active agents include, without limitation, organic molecules, inorganic materials, proteins, peptides, nucleic acids (e.g., genes, gene fragments, gene regulatory sequences, and antisense molecules), nucleoproteins, polysaccharides, glycoproteins, and lipoproteins. Classes of biologically active compounds that can be incorporated into the composition provided herein include, without limitation, anticancer agents, antibiotics, analgesics, anti-inflammatory agents, immunosuppressants, enzyme inhibitors, antihistamines, anti-convulsants, hormones, muscle relaxants, antispasmodics, ophthalmic agents, prostaglandins, anti-depressants, anti-psychotic substances, trophic factors, osteoinductive proteins, growth factors, and vaccines.

In some aspects, the additive is a therapeutic agent. As used herein, the term “therapeutic agent” means a molecule, group of molecules, complex or substance administered to an organism for diagnostic, therapeutic, preventative medical, or veterinary purposes. As used herein, the term “therapeutic agent” includes a “drug” or a “vaccine.” This term include externally and internally administered topical, localized and systemic human and animal pharmaceuticals, treatments, remedies, nutraceuticals, cosmeceuticals, biologicals, devices, diagnostics and contraceptives, including preparations useful in clinical and veterinary screening, prevention, prophylaxis, healing, wellness, detection, imaging, diagnosis, therapy, surgery, monitoring, cosmetics, prosthetics, forensics and the like. This term can also be used in reference to agriceutical, workplace, military, industrial and environmental therapeutics or remedies comprising selected molecules or selected nucleic acid sequences capable of recognizing cellular receptors, membrane receptors, hormone receptors, therapeutic receptors, microbes, viruses or selected targets comprising or capable of contacting plants, animals and/or humans. This term can also specifically include nucleic acids and compounds comprising nucleic acids that produce a therapeutic effect, for example deoxyribonucleic acid (DNA), ribonucleic acid (RNA), nucleic acid analogues (e.g., locked nucleic acid (LNA), peptide nucleic acid (PNA), xeno nucleic acid (XNA)), or mixtures or combinations thereof, including, for example, DNA nanoplexes, siRNA, microRNA, shRNA, aptamers, ribozymes, decoy nucleic acids, antisense nucleic acids, RNA activators, and the like. Generally, any therapeutic agent can be included in the composition provided herein.

The term “therapeutic agent” also includes an agent that is capable of providing a local or systemic biological, physiological, or therapeutic effect in the biological system to which it is applied. For example, the therapeutic agent can act to control infection or inflammation, enhance cell growth and tissue regeneration, control tumor growth, act as an analgesic, promote anti-cell attachment, and enhance bone growth, among other functions. Other suitable therapeutic agents can include anti-viral agents, hormones, antibodies, or therapeutic proteins. Other therapeutic agents include prodrugs, which are agents that are not biologically active when administered but, upon administration to a subject are converted to biologically active agents through metabolism or some other mechanism. Additionally, a silk-based drug delivery composition can contain one therapeutic agent or combinations of two or more therapeutic agents.

A therapeutic agent can include a wide variety of different compounds, including chemical compounds and mixtures of chemical compounds, e.g., small organic or inorganic molecules; saccharines; oligosaccharides; polysaccharides; biological macromolecules, e.g., peptides, proteins, and peptide analogs and derivatives; peptidomimetics; antibodies and antigen binding fragments thereof; nucleic acids; nucleic acid analogs and derivatives; an extract made from biological materials such as bacteria, plants, fungi, or animal cells; animal tissues; naturally occurring or synthetic compositions; and any combinations thereof. In some aspects, the therapeutic agent is a small molecule.

As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight of less than 5000 Daltons (5 kDa), preferably less than 3 kDa, still more preferably less than 2 kDa, and most preferably less than 1 kDa. In some cases it is preferred that a small molecule have a molecular weight equal to or less than 700 Daltons.

Exemplary therapeutic agents include, but are not limited to, those found in Harrison's Principles of Internal Medicine, 13th Edition, Eds. T. R. Harrison et al. McGraw-Hill N.Y., NY; Physicians' Desk Reference, 50th Edition, 1997, Oradell N.J., Medical Economics Co.; Pharmacological Basis of Therapeutics, 8th Edition, Goodman and Gilman, 1990; United States

Pharmacopeia, The National Formulary, USP XII NF XVII, 1990, the complete contents of all of which are incorporated herein by reference.

Therapeutic agents include the herein disclosed categories and specific examples. It is not intended that the category be limited by the specific examples. Those of ordinary skill in the art will recognize also numerous other compounds that fall within the categories and that are useful according to the present disclosure. Examples include a radiosensitizer, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifungal agent, a vaccine, a protein, or a nucleic acid. In a further aspect, the pharmaceutically active agent can be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetaminophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as Coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hdyrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides.

Anti-cancer agents include alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists/antagonists, endothelinA receptor antagonists, retinoic acid receptor agonists, immuno-modulators, hormonal and antihormonal agents, photodynamic agents, and tyrosine kinase inhibitors.

Antibiotics include aminoglycosides (e.g., gentamicin, tobramycin, netilmicin, streptomycin, amikacin, neomycin), bacitracin, corbapenems (e.g., imipenem/cislastatin), cephalosporins, colistin, methenamine, monobactams (e.g., aztreonam), penicillins (e.g., penicillin G, penicillinV, methicillin, natcillin, oxacillin, cloxacillin, dicloxacillin, ampicillin, amoxicillin, carbenicillin, ticarcillin, piperacillin, mezlocillin, azlocillin), polymyxin B, quinolones, and vancomycin; and bacteriostatic agents such as chloramphenicol, clindanyan, macrolides (e.g., erythromycin, azithromycin, clarithromycin), lincomyan, nitrofurantoin, sulfonamides, tetracyclines (e.g., tetracycline, doxycycline, minocycline, demeclocyline), and trimethoprim. Also included are metronidazole, fluoroquinolones, and ritampin.

Enzyme inhibitors are substances which inhibit an enzymatic reaction. Examples of enzyme inhibitors include edrophonium chloride, N-methylphysostigmine, neostigmine bromide, physostigmine sulfate, tacrine, tacrine, 1-hydroxy maleate, iodotubercidin, p-bromotetramii sole, 10-(alpha-diethylaminopropionyl)-phenothiazine hydrochloride, calmidazolium chloride, hemicholinium-3,3,5-dinitrocatechol, diacylglycerol kinase inhibitor I, diacylglycerol kinase inhibitor II, 3-phenylpropargylamine, N^{o}-monomethyl-Larginine acetate, carbidopa, 3-hydroxybenzylhydrazine, hydralazine, clorgyline, deprenyl, hydroxylamine, iproniazid phosphate, 6-MeO-tetrahydro-9H-pyrido-indole, nialamide, pargyline, quinacrine, semicarbazide, tranylcypromine, N,N-diethylaminoethyl-2,2-diphenylvalerate hydrochloride, 3-isobutyl-1-methylxanthne, papaverine, indomethacind, 2-cyclooctyl-2-hydroxyethylamine hydrochloride, 2,3-dichloro-a-methylbenzylamine (DCMB), 8,9-dichloro-2,3,4, 5-tetrahydro-1H-2-benzazepine hydrochloride, p-amino glutethimide, p-aminoglutethimide tartrate, 3-iodotyrosine, alpha-methyltyrosine, acetazolamide, dichlorphenamide, 6-hydroxy-2-benzothiazolesulfonamide, and allopurinol.

Antihistamines include pyrilamine, chlorpheniramine, and tetrahydrazoline, among others.

Anti-inflammatory agents include corticosteroids, nonsteroidal anti-inflammatory drugs (e.g., aspirin, phenylbutazone, indomethacin, sulindac, tolmetin, ibuprofen, piroxicam, and fenamates), acetaminophen, phenacetin, gold salts, chloroquine, D-Penicillamine, methotrexate colchicine, allopurinol, probenecid, and sulfinpyrazone.

Muscle relaxants include mephenesin, methocarbomal, cyclobenzaprine hydrochloride, trihexylphenidyl hydrochloride, levodopa/carbidopa, and biperiden.

Anti-spasmodics include atropine, scopolamine, oxyphenonium, and papaverine. Analgesics include aspirin, phenybutazone, idomethacin, sulindac, tolmetic, ibuprofen, piroxicam, fenamates, acetaminophen, phenacetin, morphine sulfate, codeine sulfate, meperidine, nalorphine, opioids (e.g., codeine sulfate, fentanyl citrate, hydrocodone bitartrate, loperamide, morphine sulfate, noscapine, norcodeine, normorphine, thebaine, nor-binaltorphimine, buprenorphine, chlomaltrexamine, funaltrexamione, nalbuphine, nalorphine, naloxone, naloxonazine, naltrexone, and naltrindole), procaine, lidocain, tetracaine and dibucaine.

Ophthalmic agents include sodium fluorescein, rose bengal, methacholine, adrenaline, cocaine, atropine, alpha-chymotrypsin, hyaluronidase, betaxalol, pilocarpine, timolol, timolol salts, and combinations thereof.

Prostaglandins are art recognized and are a class of naturally occurring chemically related long-chain hydroxy fatty acids that have a variety of biological effects.

Anti-depressants are substances capable of preventing or relieving depression. Examples of anti-depressants include imipramine, amitriptyline, nortriptyline, protriptyline, desipramine, amoxapine, doxepin, maprotiline, tranylcypromine, phenelzine, and isocarboxazide.

Trophic factors are factors whose continued presence improves the viability or longevity of a cell. trophic factors include, without limitation, platelet-derived growth factor (PDGP), neutrophil-activating protein, monocyte chemoattractant protein, macrophage-inflammatory protein, platelet factor, platelet basic protein, and melanoma growth stimulating activity; epidermal growth factor, transforming growth factor (alpha), fibroblast growth factor, platelet-derived endothelial cell growth factor, insulin-like growth factor, glial derived growth neurotrophic factor, ciliary neurotrophic factor, nerve growth factor, bone growth/cartilage-inducing factor (alpha and beta), bone morphogenetic proteins, interleukins (e.g., interleukin inhibitors or interleukin receptors, including interleukin 1 through interleukin 10), interferons (e.g., interferon alpha, beta and gamma), hematopoietic factors, including erythropoietin, granulocyte colony stimulating factor, macrophage colony stimulating factor and granulocyte-macrophage colony stimulating factor; tumor necrosis factors, and transforming growth factors (beta), including beta-1, beta-2, beta-3, inhibin, and activin.

Hormones include estrogens (e.g., estradiol, estrone, estriol, diethylstibestrol, quinestrol, chlorotrianisene, ethinyl estradiol, mestranol), anti-estrogens (e.g., clomiphene, tamoxifen), progestins (e.g., medroxyprogesterone, norethindrone, hydroxyprogesterone, norgestrel), antiprogestin (mifepristone), androgens (e.g, testosterone cypionate, fluoxymesterone, danazol, testolactone), anti-androgens (e.g., cyproterone acetate, flutamide), thyroid hormones (e.g., triiodothyronne, thyroxine, propylthiouracil, methimazole, and iodixode), and pituitary hormones (e.g., corticotropin, sumutotropin, oxytocin, and vasopressin). Hormones are commonly employed in hormone replacement therapy and/or for purposes of birth control. Steroid hormones, such as prednisone, are also used as immunosuppressants and anti-inflammatories.

In some aspects, the additive is an agent that stimulates tissue formation, and/or healing and regrowth of natural tissues, and any combinations thereof. Agents that increase formation of new tissues and/or stimulates healing or regrowth of native tissue at the site of injection can include, but are not limited to, fibroblast growth factor (FGF), transforming growth factor-beta (TGF-beta, platelet-derived growth factor (PDGF), epidermal growth factors (EGFs), connective tissue activated peptides (CTAPs), osteogenic factors including bone morphogenic proteins, heparin, angiotensin II (A-II) and fragments thereof, insulin-like growth factors, tumor necrosis factors, interleukins, colony stimulating factors, erythropoietin, nerve growth factors, interferons, biologically active analogs, fragments, and derivatives of such growth factors, and any combinations thereof.

In some aspects, the silk composition can further comprise at least one additional material for soft tissue augmentation, e.g., dermal filler materials, including, but not limited to, poly(methyl methacrylate) microspheres, hydroxylapatite, poly(L-lactic acid), collagen, elastin, and glycosaminoglycans, hyaluronic acid, commercial dermal filler products such as BOTOX® (from Allergan), DYSPORT®, COSMODERM®, EVOLENCE®, RADIESSE®, RESTYLANE®, JUVEDERM® (from Allergan), SCULPTRA®, PERLANE®, and CAPTIQUE®, and any combinations thereof.

In some aspects, the additive is a wound healing agent. As used herein, a “wound healing agent” is a compound or composition that actively promotes wound healing process.

Exemplary wound healing agents include, but are not limited to dexpanthenol; growth factors; enzymes, hormones; povidon-iodide; fatty acids; anti-inflammatory agents; antibiotics; antimicrobials; antiseptics; cytokines; thrombin; angalgesics; opioids; aminoxyls; furoxans; nitrosothiols; nitrates and anthocyanins; nucleosides, such as adenosine; and nucleotides, such as adenosine diphosphate (ADP) and adenosine triphosphate (ATP); neutotransmitter/neuromodulators, such as acetylcholine and 5-hydroxytryptamine (serotonin/5-HT); histamine and catecholamines, such as adrenalin and noradrenalin; lipid molecules, such as 5 sphingosine-1-phosphate and lysophosphatidic acid; amino acids, such as arginine and lysine; peptides such as the bradykinins, substance P and calcium gene-related peptide (CGRP); nitric oxide; and any combinations thereof.

In certain aspects, the active agents provided herein are immunogens. In one aspect, the immunogen is a vaccine. Most vaccines are sensitive to environmental conditions under which they are stored and/or transported. For example, freezing may increase reactogenicity (e.g., capability of causing an immunological reaction) and/or loss of potency for some vaccines (e.g., HepB, and DTaP/IPV/HIB), or cause hairline cracks in the container, leading to contamination. Further, some vaccines (e.g., BCG, Varicella, and MMR) are sensitive to heat. Many vaccines (e.g., BCG, MMR, Varicella, Meningococcal C Conjugate, and most DTaP-containing vaccines) are light sensitive. See, e.g., Galazka et al., Thermostability of vaccines, in Global Programme for Vaccines & Immunization (World Health Organization, Geneva, 1998); Peetermans et al., Stability of freeze-dried rubella virus vaccine (Cendehill strain) at various temperatures, 1 J. Biological Standardization 179 (1973). Thus, the compositions and methods provided herein also provide for stabilization of vaccines regardless of the cold chain and/or other environmental conditions.

In some aspects, the additive is a cell, e.g., a biological cell. Cells useful for incorporation into the composition can come from any source, e.g., mammalian, insect, plant, etc. In some aspects, the cell can be a human, rat or mouse cell. In general, cells to be used with the compositions provided herein can be any types of cells. In general, the cells should be viable when encapsulated within compositions. In some aspects, cells that can be used with the composition include, but are not limited to, mammalian cells (e.g. human cells, primate cells, mammalian cells, rodent cells, etc.), avian cells, fish cells, insect cells, plant cells, fungal cells, bacterial cells, and hybrid cells. In some aspects, exemplary cells that can be can be used with the compositions include platelets, activated platelets, stem cells, totipotent cells, pluripotent cells, and/or embryonic stem cells. In some aspects, exemplary cells that can be encapsulated within compositions include, but are not limited to, primary cells and/or cell lines from any tissue. For example, cardiomyocytes, myocytes, hepatocytes, keratinocytes, melanocytes, neurons, astrocytes, embryonic stem cells, adult stem cells, hematopoietic stem cells, hematopoietic cells (e.g. monocytes, neutrophils, macrophages, etc.), ameloblasts, fibroblasts, chondrocytes, osteoblasts, osteoclasts, neurons, sperm cells, egg cells, liver cells, epithelial cells from lung, epithelial cells from gut, epithelial cells from intestine, liver, epithelial cells from skin, etc, and/or hybrids thereof, can be included in the silk/platelet compositions disclosed herein. Those skilled in the art will recognize that the cells listed herein represent an exemplary, not comprehensive, list of cells. Cells can be obtained from donors (allogenic) or from recipients (autologous). Cells can be obtained, as a non-limiting example, by biopsy or other surgical means known to those skilled in the art.

In some aspects, the cell can be a genetically modified cell. A cell can be genetically modified to express and secrete a desired compound, e.g. a bioactive agent, a growth factor, differentiation factor, cytokines, and the like. Methods of genetically modifying cells for expressing and secreting compounds of interest are known in the art and easily adaptable by one of skill in the art.

Differentiated cells that have been reprogrammed into stem cells can also be used. For example, human skin cells reprogrammed into embryonic stem cells by the transduction of Oct3/4, Sox2, c-Myc and Klf4 (Junying Yu, et. al., Science, 2007, 318, 1917-1920 and Takahashi K. et. al., Cell, 2007, 131, 1-12).

The lifetime (e.g., stability) of the biologically-based ink compositions depends on the usage and the storage conditions. In some aspects, storage in a refrigerator at 4 degree C. when finishing printing is recommended. In some aspects, the biologically-based ink compositions (with our without dopants) may be stored without refrigeration, such as at room temperature (typically between about 18-26° C.) for an extended duration of time without significant loss of function. In some aspects, the biologically-based ink compositions (with our without dopants) may be stored at room temperature (typically between about 18-26° C.) for an extended duration of time, such as at least for 1 week, at least for 2 weeks, at least for 3 weeks, at least for 4 weeks, at least for 6 weeks, at least for 2 months, at least for 3 months, at least for 4 months, at least for 5 months, at least for 6 months, at least for 9 months, at least for 12 months, at least for 15 months, at least for 18 months, and at least for 24 months, or longer, without significant loss of function.

In some aspects, the biologically-based ink compositions (with or without dopants) may be stored at elevated temperature (between about 27-40° C.) for at least part of the duration of storage, for an extended duration of time, such as at least for 1 week, at least for 2 weeks, at least for 3 weeks, at least for 4 weeks, at least for 6 weeks, at least for 2 months, at least for 3 months, at least for 4 months, at least for 5 months, at least for 6 months, at least for 9 months, at least for 12 months, at least for 15 months, at least for 18 months, and at least for 24 months, or longer, without significant loss of function.

In some aspects, the present disclosure provides water-based, biologically-based ink compositions that can be transferred onto everyday use objects and parts using different techniques. The techniques described allow for new methods of printing biologically-based ink compositions on different substrates, including substrates of a large scale. The biologically-based ink compositions can be used to functionalize soft and conformal substrates like paper and textiles, as well as hard ones such as plastic, ceramic, or metal, to make objects dynamically respond to changes in surrounding environment.

The ink and paste compositions provided herein are water based. They are environmentally friendly and they avoid the use of harsh chemicals in the making process allowing their use in any type of setting ranging from laboratories to art studios.

The ink compositions are based on diverse combinations of biomaterials processed using environmental friendly approaches at room temperature, controlled pH conditions (range 5-8), and concentration ranges spanning between 2-6 wt %. These properties are tuned depending on the ink function, machinery specifications, and substrate geometry or surface conditions.

Proteins are by nature macromolecules, and consequently the viscosity of their solutions is often dramatically affected by changes in concentration. At higher concentrations, the capillary force is insufficient to break the filament of the droplet during the ejection, and the droplet retracts back into the nozzle during various printing techniques. For polymers, the micro-rheological explanation for this behavior is that the coiled and folded polymer chains are elongated in the direction of flow into a stretched state, which is accompanied by a strong increase of the hydrodynamic drag. However, most proteins, unlike synthetic linear polymers, are not randomly coiled chains; rather, most proteins tend to be carefully folded into organized structures in their native state, and the degree to which proteins are either globular or fibrous plays an important role in their intrinsic viscosity, which, consequently affects the maximum concentration of a printable solution.

This implies a relative facility in the printing of globular protein such as enzymes, messenger/signaling, and transport proteins, while structural fibrous protein, such as keratin, collagen, and elastin are impossible to print at relevant concentrations or in mild conditions (e.g., neutral pH, aqueous solution). This has a significant impact on the concentration limitation of printable solutions of specific categories of important proteins, e.g., structural proteins. As an example, globular proteins may be easily printable in concentrations of 10 wt % or more with common dampened nozzles, but type I collagen solutions, for example, in concentrations even as low as 0.3-0.5 wt % (a range commonly used for biomedical applications) are unprintable with the same devices.

While a common technique for improving the printability of viscous inks is to raise the printing temperature, there are practical restrictions which further limit the printability of structural proteins.

Bioprinting, in some processes, involve shear rates in the range of 2×10⁴ to 2×10⁶ s⁻¹; while such shear rates pose no foreseeable problems for small, globular proteins, they are sufficiently high to compromise the structural integrity of some of their more fragile, larger, counterparts (e.g. structural proteins). Another hindrance in bioprinting structural proteins is the high compression rates used to generate droplets, which may result in the loss of both structural and biological properties, particularly in the absence of stabilizing additives.

The biologically-based ink compositions provided herein may be tuned for the use in various printing techniques, such as inkjet printing, extrusion-based printing, screen-printing, spray-coating and tape-layering. In some aspects, the viscosity of the inks may be is tuned to the requirements of each fabrication method. For example, the viscosities of the inks used to functionalize surfaces via screen-printing may be in the range of 1,000-10,000 cPs. The viscosities of paste-like inks used to make objects via computer-controlled extrusion systems are in the range of 50 cPs to 500 cPs. In contrast, previous inks have had viscosities in the range of 1-20 cPs. Different additives may be including in the compositions to achieve these higher viscosities in order to utilize different printing techniques.

In some aspects, viscosity may be measured by a viscometer or rheometer. Kinematic viscosity may be measured using a glass capillary viscometer. Viscosity may be measured by measuring the efflux time of the composition from a cup such as a Zahn cup or a Ford viscosity cup.

In some aspects, the biologically-based ink compositions may be inkjet printed. Inkjet printing (IJP) is an easy, inexpensive and widely accessible technology spread around the world for several decades. The fortune of IJP is tied to the pervasiveness of personal computing, as for the last two decades it has represented one of the fundamental accessories for any PC workstation. IJP is based on the use of electrical actuators to eject picoliter (pL) volumes of liquid from micrometer-wide nozzles onto a substrate in a defined pattern. IJP has gained extensive acceptance in microfabrication for basic patterning and rapid fabrication. While the most popular purpose of IJP technology remains printing paper documents, it has also been applied in organic electronics, chemical synthesis, sensor fabrication, combinatorial chemistry and biology.

Inkjet printing can be divided into two categories: (1) drop-on-demand (DoD) or impulse inkjet, where droplets are generated when required; and (2) continuous inkjet, in which droplets are deflected from a continuous stream to a substrate when needed. Inkjet printing can be further subdivided according to the specific means of generating droplets, such as piezoelectric, thermal and electrostatic. Each of these techniques has specific ranges of operation that limit their applicability. Such variables include: operating temperature range, material throughput, reproducibility of droplets, precision of deposition, range of printable viscosities, range of shear forces within the nozzle, reservoir volume and the number of fluids that may be printed during at the same time. Droplet size involves, typically, volumes ranging from 1.5 pL to 5 nL at a rate of 0-25 kHz for drop-on-demand printers (and up to 1 MHz for continuous printheads).

In some aspects, the biologically-based ink compositions may be electrohydrodynamically jet printed. Electrohydrodynamic jet printing (EHJP) can produce features as small as 1 μm wide lines, which is typically an order of magnitude smaller than inkjet printing. Naturally, the droplets produced by this technique are also smaller, being in the femto-liter region. Such small droplet sizes are of interest since this means that less material can be dispensed with more spatial control, which couples with the ongoing miniaturization seen in many applications. An open question to be addressed is whether the EHJP droplet ejection method affects the material contained within the ink. Whereas inkjet printers eject their droplets from within the nozzle, EHJ printers eject their droplets from outside the nozzle. The ink in an EHJ printer forms a droplet that is attached to the nozzle. This dome of ink is charged by a wire contained within the nozzle using voltages up to 200 V, which is necessary to overcome the surface tension and causes a Taylor cone to form. The droplets are ejected from the tip of the cone. This process likely makes protein susceptible to electrical breakdown and droplet deflection during application of inks to substrates. Furthermore, the EHJP process is still in its infancy and it has not yet been applied to the full range of applications that inkjet printing has. In addition, EHJP is based on electrostatic forces; meaning that the substrate must be conductive, which is also a limitation. Finally, the cost of the technique is another factor to consider.

In some aspects, the biologically-based ink compositions may be extrusion-based printed. Extrusion-based printing has been widely used in the metal and plastics industries for shape forming different materials and 3D-printing. Extrusion-based printing employs a fluid-dispensing system through an extruder or nozzle to shape form materials and deposit the extruded materials layer-by-layer onto a substrate. Extrusion-based printing systems are computer-controlled to deposit materials layer-by-layer to form 3D structures. Extrusion-based printing systems may operate under a robotic control system.

Computer-controlled extrusion printing allows the realization of 2D and 3D objects with sizes ranging from millimeters up to meters. Computer controlled extrusion is different than inkjet printing in: scale, types of printable inks, and result configurations. Computer controlled extrusion utilize gantries which move the extruder in a direction required to deposit a certain pattern. Gantries may be robotically controlled arms and may include end effectors capable of holding or manipulating tools and substrates. Gantry size can be much larger in extrusion printing, expanding the print bed to meter scales. Inks can be much varied as extrusion technology allows for a wide range of viscosities from liquids, to colloids, to thick pastes. For example, the viscosities of inks used to make objects via computer-controlled extrusion systems are in the range of 50 cPs to 500 cPs.

In contrast, ink jet printing allows for liquid drop by drop deposition only. Ink jet printing is limited to very thin applications onto substrates such as paper and fabric. In extrusion printing, inks can be thinly applied onto substrates, form thick layers onto substrates, be extruded in 2D without a substrate, and also form 3D objects similar to layer by layer depositions observed in 3D fused deposition printing.

In some aspects, the biologically-based ink compositions may be spray coated. Spray coating is the application of one or more thin layers of material onto a substrate. Inks in liquid or gas state are sprayed from a reservoir via a nozzle, and left to dry onto the substrate. Systems for spray coating may be computer-controlled systems or use robotically controlled system components. Systems for spray coating may be manually actuated or powered and may contain one or more nozzles. The shape and size of the nozzle may vary and depend on the application. Spray coating techniques also allow for a larger surface area to be printed and depending on the ink composition, functionalized.

In some aspects, the biologically-based ink compositions may be screen printed. Screen printing is a technique where a mesh is used to transfer ink onto a substrate, except in areas made impermeable to the ink by a blocking stencil. In one example, textiles are used as a substrate for screen-printing. Textiles are placed on flat substrates. Custom-designed screens loaded with ink compositions are laid on top of them. A squeegee is used to transfer the ink onto the underlying fabric by applying a constant pressure. The screen-printed substrates may be left to dry at room temperature.

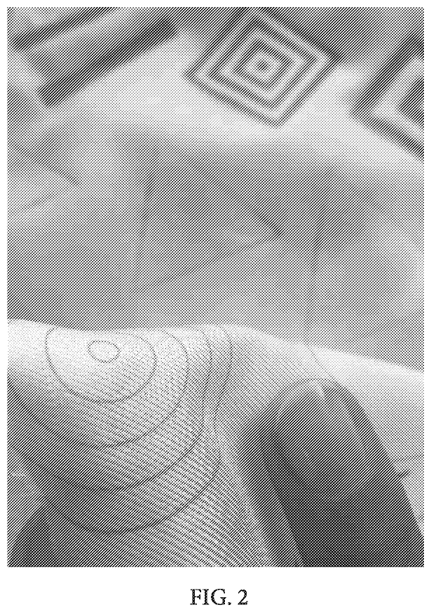

The viscosities of the ink compositions used with screen-printing techniques are in the range 1,000-10,000 cPs. Screen printing allows the functionalization of surfaces that range from tens of millimeters up to tens of meters. The minimum resolution of the transferred features spans between tens of micron up to hundreds of millimeters. In one example, pH sensing fabrics can be implemented via screen printing so that materials are transferred into one dimensional substrates. For instance, objects sensitive to temperature variations can be developed via computer-controlled extrusion printing in order to generate two and three dimensional objects (see FIG. 2).

In some aspects, the biologically-based ink compositions may be tape layered. Automated tape laying is a technique where tape is mechanically applied by a tape pressing end-effector onto a mold, creating a multi-layered surface while ensuring that individual layers are oriented in different directions.

In some aspects, the biologically-based ink compositions may be bio-printed. Bio-printing has been defined in the context of inkjet printing biomaterials. The field of bio-printing originated in the mid-1990s, with an expansion after the turn in the new millennium. Several factors contributed to this increase: by 1985 functional materials for sensing biological matter (e.g. for glucose and urea) were spatially disposed to form multi-analyte arrays. In 1987 the first patent for an inkjet-printed enzyme-based biosensor was filed. The robustness and versatility of inkjet printing enabled researchers to modify the technique to meet their needs. In particular, the mild conditions afforded by the IJP process make it particularly suited for handling biological materials. Minimal sample contamination and waste together with the accurate control and placement of pre-determined quantities of material are also highly appealing features. The possibility to integrate biomaterials with IJP technology is convenient because it combines ease, low-cost and robustness. In particular, the use of IJP to deposit proteins is of particular appeal given that the impossibility to apply conventional polymer processing techniques to proteins is one of the major hindrance to their applications as biomaterials.

Extrusion-based printing has been used in the field of tissue engineering for fabrication of living tissues and is referred to as extrusion-based bioprinting (EBB) (Ozbolat et al. Biomaterials; 2015). Extrusion based-printing and screen-printing allow for the printing on larger substrates or forming larger objects and formulations of biologically-based ink compositions formulated for these methods are provided herein.

In some aspects, inkjet printing can be employed for printing patterns of the biologically-based ink compositions, such as silk fibroin inks (with or without dopants), onto a substrate. The printable patterns (e.g., structures) using the biologically-based ink compositions are limitless, simply depending on the printing technique. The printable patterns (e.g., structures) include, but are not limited to regular and irregular patterns, such as lines, curves, dots, solids, and any combinations thereof. In some aspects, the pattern may form a microfluidic channel, which may be used for drug diagnostics.

Each pattern or structure to be printed is formed from a plurality of small “dots” each of which is generated from a liquid droplet of the biologically-based ink compositions deposited onto the substrate. Such patterns can be either one layer of dot prints or multilayer of prints (e.g., serial printing), depending on the intended applications. Each layer in the multilayer prints can be overlapping on top of each other for thicker patterns or cross with other layers for complicated patterns. In some aspects, serial printing can be performed to fabricate a 3D structure.

Compositions of the biologically-based ink compositions provided herein allow for the use of additional printing techniques such as extrusion-based printing and screen-printing which allow for printed patterns on the millimeter to meter scale. These patterns may vary depending on the substrate and their functionality and may be preprogrammed in the process of computer-controlled extrusion, or pre-designed on screens for screen-printing. The patterns may expand across the entire surface of the substrate.

A variety of substrates may be suitable for use in printing the biologically-based ink compositions provided herein. Such printable substrates using the biologically-based ink compositions are limitless, depend on the formulation of the composition, the type of printing methods being used, and the desired end functionality of the printed substrate. Non-limiting examples of useful substrates include, but are not limited to: papers, polyimide, polyethylene, natural fabric, synthetic fabric, silk fabric, silk fibers, silk threads, porous silk, metals, liquid crystal polymer, palladium, glass and other insulators, silicon and other semiconductors, metals, cloth textiles and fabrics, plastics, biological substrates, such as cells and tissues, protein- or biopolymer-based substrates (e.g., agarose, collagen, gelatin, etc.), wood, ceramic and any combinations thereof.

Substrates may be 2- or 3-dimensional, both soft and hard materials. The ink may interact with the substrate's surface first and can penetrate other layers of the substrate, for example, if the substrate is made of a porous, water-permeable material.

In some aspects, the biologically-based ink compositions can be printed on substrates that generally are of a flexible material, such as a flexible polymer film or paper, such as wax paper or non-wax substrates. In some aspects, the biologically-based ink compositions can be printed on an elastic polymer. In some aspects, suitable substrates include releasable substrates, such as a label release grade or other polymer coated paper, as is known in the art (e.g., see U.S. Pat. No. 6,939,576). Such substrate also can be or include a non-silicone release layer. Such substrate also can be a plastic or polymer film, such as anyone of an acrylic-based film, a polyamide-based film, a polyester-based film, a polyolefin-based film such as polyethylene and polypropylene, a polyethylene naphthylene-based film, a polyethylene terephthalate-based film, a polyurethane-based film or a PVC-based film, or a combination thereof.

The present disclosure encompasses the use of substrates which have ranges of different stiffnesses. A flexible substrate may conform with the body and be configured to function as a biometric sensor.

The scale of the substrate can be very varied, as the scale of printing depends on the technique chosen, from nano- to macro-scale via, for instance, inkjet printing, or via, for instance, industrial robotic manufacturing methods.

End-effectors in industrial-scale manufacturing are the devices at the end of machinery designed to interact with the environment. The ones that can apply or build with our functional inks include: brushes, spray guns, extrusion syringes, fused deposition nozzles, automatic squeegee handles etc.

In some aspects, the additives, dopants, and biologically active agents for use with the biologically-based ink compositions can be used in any of the above fabrication methods due to the ability to tune the inks to the diverse platform-specific properties, such as modifying the viscosity using additives, fillers, and using different biomaterial extraction methods.

Compositions of the biologically-based ink compositions provided herein allow for the use of additional printing techniques such as extrusion-based printing and screen-printing which allow for printed patterns on the millimeter to meter scale. These patterns may vary depending on the substrate and their functionality and may be preprogrammed in the process of computer-controlled extrusion, or pre-designed on screens for screen-printing.

Resolution of the patterns may vary depending on the fabrication methods, the composition of the biologically-based ink and the type of substrate. Surface properties, specifically of the substrate can limit the resolution of the print and include properties such as roughness.

In some aspects, apparatus, devices and/or objects include the biologically-based ink compositions printed on substrates may be utilized to functionalize a substrate, for example, into an environmental stimuli sensor. Environmental stimuli may include pH variations, such as in sweat, rain or gas pollution. Other environmental stimuli may include temperature, or the ability to sense contaminants such as bacteria or toxic spills. Conductive ink compositions may be used to form printed biosensors. Printed substrates for sensing environmental stimuli may include textiles and wearable apparel such as gloves, furniture and wall art.

Various printing techniques, types of substrates and ink compositions have been provided herein. Different combinations of these elements produce different aspects of functional devices using the biologically-based ink compositions printed on substrates.

Ink compositions printed on substrates may be utilized to functionalize a substrate into an environmental stimuli sensor. In one aspect, ink compositions are screen-printed on to textiles. The ink compositions can contain additives which are sensitive to environment conditions. For example, pH-sensitive dyes may be used as additives to the ink compositions. Once applied in a pattern to a substrate, such as a textile, the pattern may change color in response to a variation in pH.

Colorimetric sensing ink compositions can be used in a variety of printing techniques, resulting in different devices. For instance, pH sensing fabrics can be implemented via screen printing so that materials are transferred into one dimensional substrates. Variations in pH may be detected in for example, sweat, rain or gas pollution.

Other environmental stimuli may include temperature, or the ability to sense contaminants such as bacteria or toxic spills. Ink compositions may contain additives which are able to sense air quality. Contamination in an environment may be detected by using various molecules such as enzymes, vaccines, drugs, and antibodies to detect various target analytes such as heavy metals in water and the presence of parasites in biological fluids such as blood.

In some aspects, objects sensitive to environmental stimuli can be developed via computer-controlled extrusion printing in order to generate two and three dimensional objects. Substrates may include but are not limited to any of the materials previously described herein and may range in size from a few millimeters to greater than a meter. Printed substrates for sensing environmental stimuli may include textiles and wearable apparel such as gloves, furniture and wall art.

In some aspects, electrochemical sensors may be created by screen-printing onto large substrates such as textiles. Conductive traces using graphene-based conductive ink compositions are formulated and used as conductive traces. The conductive traces may act as biosensors detecting biometric signals or environmental stimuli.

Using a layer by layer deposition done through screen-printing or extrusion-based printing, microfluidic channels can be built using the biologically-based ink compositions. This includes patterning hydrophobic and hydrophilic areas on fabric. When the printed substrate is contacted with water, the hydrophilic regions will dissolve and the fluid will be passively drawn only in certain directions. Separate regions on the substrate may be maintained for analysis. Regions where the fluid is drawn may contain sensing regions to sense different properties of the fluid.

The present disclosure describes formulations of biologically-based ink compositions and methods of printing that incorporate functionalities into substrates of large formats, moving beyond the lab-scale that usually confines these types of applications to few centimeters. The biologically-based ink compositions can be used to implement non-toxic conductive inks and they can embed, and successfully preserve, scent and flavor, pH sensitive dyes, active bio-molecules such as enzymes, vaccines, drugs, and antibodies to detect various target analytes such as heavy metals in water, the presence of parasites in biological fluids such as blood, without the troublesome use of hydration or refrigeration. These formulations enable a new kind of biologically, environmentally, and technologically connected substrates. These printed substrate provide not only new modalities for wearable sensors, therapeutics or interactive devices, but also offer new aesthetic avenues for design and development.

Several difficulties have previously been encountered in designing, producing, and tuning these devices are ultimately hampering their transformation from lab bench prototypes into real world products. Prior inks had a water-like viscosity and they could only transferred by drop-by-drop deposition systems using, i.e., inkjet printing on flat substrates defined by confined, small-scale printed areas as disclosed in previous inventions (WO2014/085725). Ink-jet printing is achieved by propelling droplets of ink onto paper, plastic, or other substrates, and it is limited to liquid inks and drop by drop deposition. Traditional ink-jet printing allows the functionalization with features on the order of micrometers on substrates that are 70×90 cm. Scaling-up to print functional inks on surfaces that have meter sizes (e.g. wall-paper in buildings) is challenging. Objects of such a scale are absent from the scientific literature or from the consumer market. They are now feasible with our invention using computer-aided extrusion printing, screen printing and a combination of both, because we are able to tune our functional inks to the material and mechanical requirements of these fabrication methods at scale.

One difference between the ink compositions of the present disclosure and previous cases is the use of regenerated silk solutions in combination with other biomaterials to realize customized pastes depending on the printing technique of election. The present disclosure discloses variable viscosity inks to make both small and large-scale objects.

The viscosity is tightly related to the “printability” of the ink compositions, and it is tuned to the requirements of each fabrication method. The viscosities of the ink compositions provided herein may be used to functionalize surfaces may be in the range of 50 cPs to 500 cPs. They match the standard viscosities (e.g. 1,000-10,000 cPs) of commercially available inks for screen-printing. The viscosities of paste-like inks used to make objects via computer-controlled extrusion systems are in the range of 50 cPs to 500 cPs. The inks and pastes here described may be water based. They are environmental friendly and they avoid the use of harsh chemicals in the making process allowing their use in any type of setting ranging from laboratories to art studios.

Pastes for computer-aided extrusion and screen-printing are made of a thickening agent (e.g. sodium alginate or guar gum), a binding agent (e.g. silk) and a plasticizer (e.g. glycerol). Their combination relies on the viscosity needed and on the printing technique used (i.e. extrusion vs screen printing). The printing pastes are also tuned depending on the substrate of choice (e.g. plastics, paper, textiles, wood) by choosing different binding agents depending on the base matrix being regenerated silk fibroin, and regenerated cellulose solutions.

Computer controlled extrusion is different than inkjet printing in: scale, types of printable inks, and result configurations. Gantry size can be much larger in extrusion printing, expanding the print bed to meter scales. End-effectors of industrial-scale fabrication machinery such as robotic arms may be used to move components such as the extruder in order to print large scale objects. Ink compositions may be varied as extrusion technology allows for a wide range of viscosities from liquids, to colloids, to thick pastes. In contrast, ink jet printing allows for liquid drop by drop deposition only. Ink jet printing is limited to very thin applications onto substrates such as paper and fabric. In extrusion printing, inks can be thinly applied onto substrates, form thick layers onto substrates, be extruded in 2D without a substrate, and also form 3D objects similar to layer by layer depositions observed in 3D fused deposition printing. Pressure-based extrusion printing allows sub-millimeter features depending on syringe nozzle size, and can form meter-scale 2D or 3D objects with or without the use of substrates.

Screen-printing allows transferring features that are in the range of tens of microns, depending on screen mesh size and ink particle size, onto substrates that can be of tens of meters long and wide. Screen printing allows the functionalization of surfaces that range from tens of millimeters up to tens of meters. The minimum resolution of the transferred features spans between tens of micron up to hundreds of millimeters.

This present disclosure allows for the use of ink compositions in large format designs and provides fully integrated objects and parts able to sense and monitor physical and chemical variations in both local and global environments. New sensing function is incorporated into large format designs, moving beyond the lab-scale that usually confines these types of applications to few centimeters, and potentially enable a new kind of biologically, environmentally, and technologically connected objects. Versatile sensing surfaces can now be obtained and opportunely tuned for different applications by adapting the chemical processes for embedding and preserving the active biomolecules (e.g. drugs, antibodies, enzymes), without need for hydration and refrigeration. Sensitive areas able to simultaneously monitor multiple analytes can now be implemented within daily used products. Sensing areas designed on these substrates may be opportunely tuned with biomaterials able to drive the fluid to be detected in specific directions. These substrates are in close contact with the surface to be monitored, integrating both sensing and harvesting on the same support material. The techniques provided herein are instrumental for the wide diffusion and adoption of active surfaces that are able to sense, monitor, interact and actively respond to chemical stimuli.

### EXAMPLES

Although the present invention has been described with reference to particular examples and aspects, it should be understood that the present invention is not limited to those examples and aspects. Moreover, the features of the particular examples and aspects may be used in any combination. The present invention therefore includes variations from the various examples and aspects described herein, as will be apparent to one of skill in the art.

### Example 1—Exemplary Materials and Methods for Ink Compositions

Materials. Silk cocoons of Bombix mori silkworm were purchased from Tajima Shoji (Japan). Sodium carbonate, lithium bromide, phenol red sodium salt, bromocresol green sodium salt, nitrazine yellow, glycerol, sodium alginate, chitosan, methanol and acetic acid were purchased from Sigma-Aldrich (USA). Thermochromic pigments were purchased from Atlanta Chemicals (USA). All chemicals were used as received and they followed trace metal standard, when possible. Deionized water with resistivity of 18.2 MΩ cm was obtained with a Milli-Q reagent-grade water system and used to prepare aqueous solutions. Ahlstrom paper Grade 55 from VWR (USA) and Whatman Paper Grade 4 from Fisher Scientific (USA), silk from B&J Fabrics (New York, USA) were employed as substrates for inkjet printing and screen-printing.

Silk fibroin solution preparation. Silk fibroin was extracted boiling finely chopped Bombix mori silk cocoons in a solution of 0.02 M sodium carbonate to remove the hydrophilic layer of sericin for 30 minutes (inks for screen printing) or 2 hours (inks for inkjet printing). The fibers were washed three times for 20 minutes in deionized water and dried overnight. They were dissolved in a solution of lithium bromide (9.3 M) at 60° C. for 4 hours. A 20 wt % solution was obtained and dialyzed against deionized water for 2 days. The final solution was centrifuged twice at a speed of 9000 rpm at 4° C. for 20 minutes and then filtered to obtain a 7-8 wt % silk fibroin solution.

Ink Compositions for inkjet printing. Ink compositions for inkjet printing were realized diluting silk fibroin down to a final concentration of 4 wt %. pH indicators (2.5 mg/mL) were added one at time and mixed to obtain an homogeneous, colored solution of silk fibroin that was stored in the fridge between uses. Dimatix Materials Cartridges were filled and 4 layers were inkjet printed on paper substrates using the Dimatix DMP 2800 from Fujifilm, Santa Clara, Calif., USA. The substrates were left to dry for 12 hours before use or stored at room temperature in a dark environment.

Ink Compositions for screen-printing. Ink compositions for screen-printing were realized mixing glycerol and sodium alginate (4 wt %) in a weight ratio of 1:2, 5 mg/ml of pH indicator, and a solution of silk fibroin with a final concentration of 2 wt %. Three biologically-based ink samples were prepared with pH indicators of Bromocresol Green (BG), Nitrazine Yellow (NY), and Phenol Red (PR), and the rheology of the inks were characterized via steady state shear tests to evaluate the printability of the inks. The biologically-based inks were printed through a mesh screen at a shear rate of 100-200 s⁻¹. In the shear rate range of 100-200 s⁻¹, viscosity η decreases from 34.6 P down to 14.8 P for BG inks, from 35.3 P down to 12.8 P for NY inks, and from 29.3 P down to 16.3 P for PR inks. The type of pH indicator employed does not affect the rheological properties of the biomaterial-based inks and they match their commercial counterparts. This is shown by viscosity η decreasing from 27.7 P down to 15.1 P in the shear rate range of 100-200 s⁻¹. In summary, the biomaterial-based inks presented here can be successfully screen-printed and scaled-up for mass production.

The biologically-based ink compositions were printed onto wearable substrates, such as cotton, paper, or crepe de chine silk fabric. Protocols from the ASTM Standard D5035-11(2015), which is incorporated by reference in its entirety herein, are adapted to estimate the impact of ink printing on the overall tensile strength and elongation at break of the textiles.

Average stress-stretch curves of bare silk fabric substrates and screen-printed silk fabric substrates embedding three different pH indicators were developed and tested. The plots reveal differences especially at small deformations (λ=1.00-1.04), where the bare substrates are more compliant with an average stress-stretch slope of 18.5±0.1 MPa (n=5).

Under similar conditions (λ=1.00-1.04), screen-printed substrates are stiffer, and exhibit average stress-stretch slopes of 73.1±0.4 MPa (n=5) for BG, 75.1±0.4 MPa (n=5) for NY, and 90.3±0.6 MPa (n=5) for PR, respectively. At larger extensions (i.e., from λ=1.15 up to the beginning of failure), all substrates behave similarly as they exhibit a stiffer quasi-linear behavior. Average stress-stretch slopes at λ=1.15 are 178±2 MPa for bare silk and range between 183±3 MPa and 201±3 MPa for screen-printed substrates. The bare substrates start to break at λ=1.38, which correspond to an average failure strength of 47.4±4.5 MPa (n=5). Printed substrates start to break at similar stretch levels but reach higher failure strengths. Substrates printed with inks embedding BG rupture at λ=0.38 (failure strength=56.1±1.9 MPa (n=5)), while those embedding NY rupture at λ=0.43 (failure strength of 62.4±1 MPa (n=5)) and at λ=0.37 (failure strength of 54±7.3 MPa (n=5)) for PR.

The sensing textiles embedding BG and NY are able to discriminate 0.1 and 0.2 units of pH, respectively, while those embedding PR are able to discriminate 0.2 and 0.5 units for the first and second linear range. To the best of the Applicants knowledge, obtaining resolutions of 0.1 units with a functionalized sensing textile is an achievement first of its kind that opens up pathways towards the detection and discrimination of at least 0.5 units of pH by tuning the biomaterial-based inks here introduced.

Demonstrated herein, active biologically-based inks are screen-printed on textiles in a highly reproducible fashion. The functionalized areas are on the order of square meters, with resolutions down to hundreds of μm, and with multispectral combination of colors allowing distributed mapping of different environments they get in contact with. These results are first of their kind and they are appealing for environmental and biological real-time monitoring of pH variations.

### Example 2—Exemplary Production of Substrates with Hydrophilic and Hydrophobic Regions

Hydrophobic regions can be designed and realized through spray-coating or screen-printing commercial water repellent materials. It is possible to guide the fluid into different directions depending on the design.

In one example, hydrophobic and hydrophilic regions are created by transferring commercial water repellent materials onto a substrate. Sensing regions can be screen-printed using hydrophilic inks in the remaining regions not yet covered by the hydrophobic material. Hydrophobic and hydrophilic regions create microfluidic channels and may be built using layer by layer deposition done through screen-printing. Screen printing of ink compositions for screen-printing may be formulated using the methods of Example 1.

Microfluidic channels can be designed by patterning hydrophobic and hydrophilic areas, maintaining separate regions for analysis. After being in contact with water, the hydrophilic regions will dissolve and the fluid will be passively drawn only in certain directions. Areas are formed that can direct the liquid towards localized, sensitive regions on the substrate. Substrates may be any substrate for example, paper, plastic and textiles. Sensitive regions may be regions functionalized as sensors to sensor environmental stimuli.

Testing is conducted to verify wetting and non-wetting properties of the textiles with the printed microfluidic channels.

### Example 3—Exemplary Production of Graphene-Based Conductive Inks on Substrates

Graphene-based conductive ink compositions are made and used as conductive traces.

Graphite is exfoliated in a sodium alginate/water based solution to obtain a graphene based conductive ink composition. The consistency and conductivity of ink compositions is optimized for the type of printing being done and the type of sensor being made. In one example, conductive traces are produced by screen-printing.

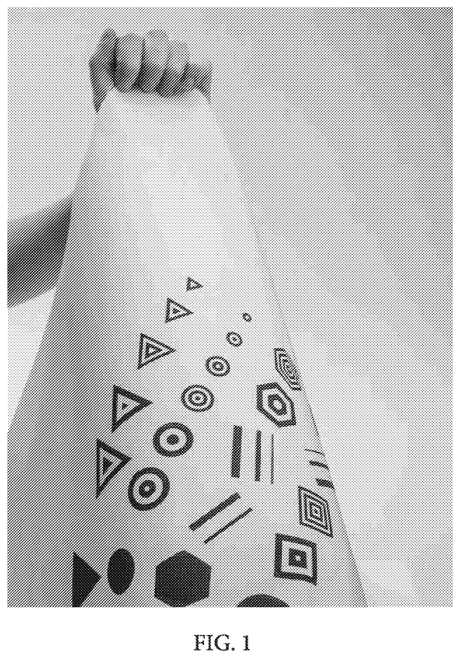

The transfer of functionalized inks based on biomaterials (i.e. made out of silk and sodium alginate) onto flexible substrates such as silk fabrics in one example, is done by screen-printing. FIG. 1 shows a centimeter-scale soft functional object which includes technical apparel having conductive traces consisting of a non-toxic conductive ink compositions with a green chemistry approach for technical apparel and fabric integrated wearable devices. The graphene conductive ink composition is transferred on to a substrate which may be plastic, paper and fabric substrates. Conductive ink compositions based on biomaterials will be produced using green chemistry procedures.

Mechanical and electrical tests are used to test the function of the conductive patterns as sensors when transferred onto plastic, paper and fabric.

### Example 4—Exemplary Methods of Screen Printing with Silk-Based Ink Compositions

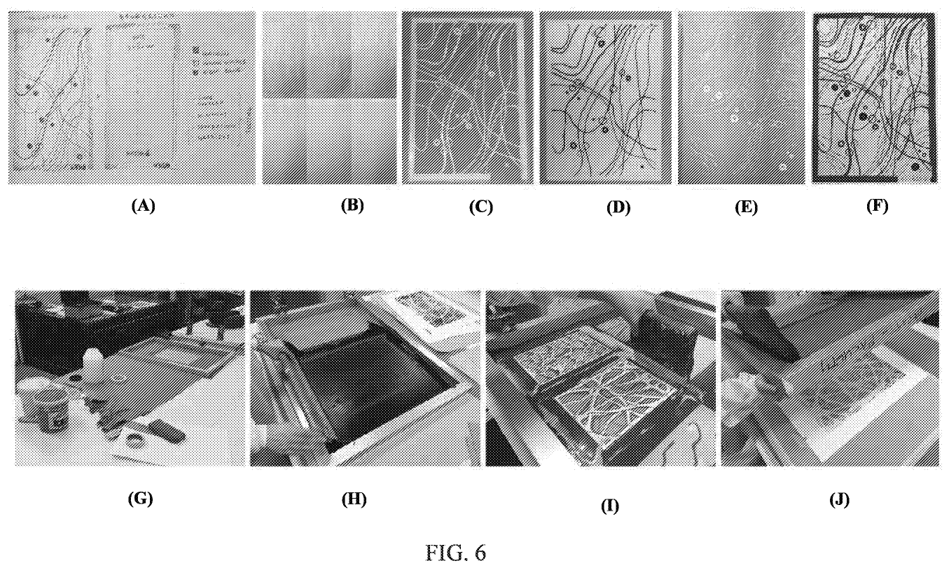

Drawings are used to optimize printing parameters such as patterns, dimensions, resolution and screen formats as shown in FIG. 6 panel (A) and panel (B). Patterns are transferred on to the screen as shown FIG. 6 panel (C), panel (D), panel (E), and panel (F). The patterns are designed depending on the final application for example, if the final printed substrate is configured as a chemical sensor, microfluidic channels, or conductive traces.

Inks based on silk and alginate biomaterial mixtures are produced. Their composition and viscosity properties are optimized via screen-printing on different substrates. For example, the substrate may be paper or textiles such as silk fabrics. Fabrics may be characterized as having different stiffness. The process of screen-printing on a substrate is shown in FIG. 6 including the materials for transferring the ink to a screen in panel (G), applying the ink to a screen in panel (H), and using a squeegee to apply the ink composition to a substrate in panel (I). The printed substrate is then removed from the screen and allowed to dry as shown in panel (J). The resolution that characterizes the images and that is transferred on the various substrates will depend on, among other variables, the ink composition and the type of substrate used.

### Example 5—Silk-Based Printed Ink Compositions for pH Sensing

Silk-based ink compositions containing sodium alginate can be embedded with molecules able to detect various analytes. For example, a commercial pH sensitive dye is incorporated into the ink composition. The color of the ink will change depending on the pH of the solution they will be exposed to. The ink compositions may be printed in a pattern onto substrates including but not limited to paper, transparent plastic films, and silk fabrics.

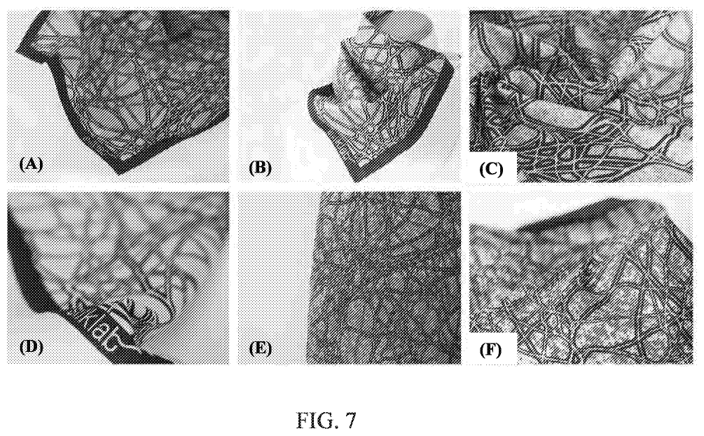

Colorimetric sensing ink compositions can be used in a variety of printing techniques, resulting in different devices. In this example, ink compositions are transferred onto one dimensional substrates. FIG. 7 panel (A), panel (B), panel (C), panel (D), panel (E), and panel (F) show examples of pH sensing fabrics that can be implemented using screen printing.

Patterns may be printed on different scales. The patterns may be printed on a millimeter-scale. For example, ink compositions are screen-printed onto a haute-couture scarf made from silk fabric. The color of the ink composition indicates variation in pH variations. In other aspects, the ink composition may contain a molecule capable of detecting sweat, rain, or gas pollution.

### Example 6—Silk-Based Printed Ink Compositions for Sensing Environmental Stimuli

Colorimetric sensing inks can be used in a variety of printing techniques, resulting in different devices or functional objects. Functional inks can be tuned depending on the substrate and on the fabrication method of choice. For example, regenerated silk can be embedded within functional inks for screen-printing on silk substrates to improve the binding of the ink.

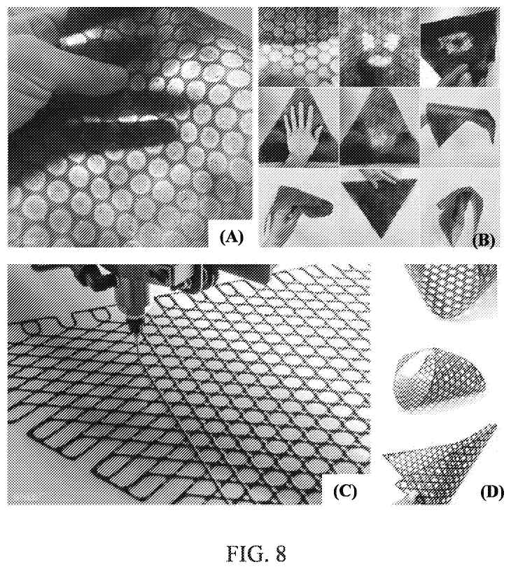

In one example, objects sensitive to temperature variations can be developed via computer-controlled extrusion printing in order to generate two and three dimensional objects as shown in FIG. 8 panel (A)-panel (D). Inks are thickened so that their sheer thinning is tuned to the requirements of pressure-based extrusion printing method as shown in FIG. 8.

In another example, inks are able to sense contaminants such as bacteria, or toxic spills. Inks may be printed on different types of substrates including the example shown in FIG. 2 that shows a decimeter-scale soft functional object including lightweight furniture which has been printing using directional tensile screen-printed fabric for lounge furniture.

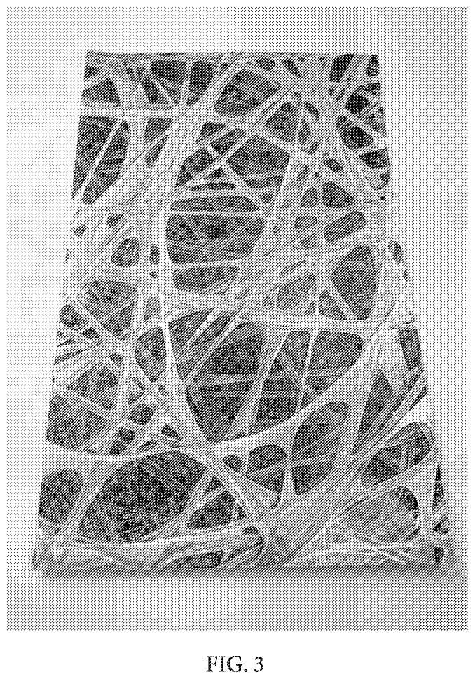

In another example, an ink composition printed on a substrate is able to sense air quality. FIG. 3 shows meter-scale soft functional wall paper which has been screen-printed with silk-based ink compositions using CMYK (cyan, magenta, yellow, and key (black)) color separation. The silk-based ink compositions forming wall art is functionalized with molecules able to sense air quality.

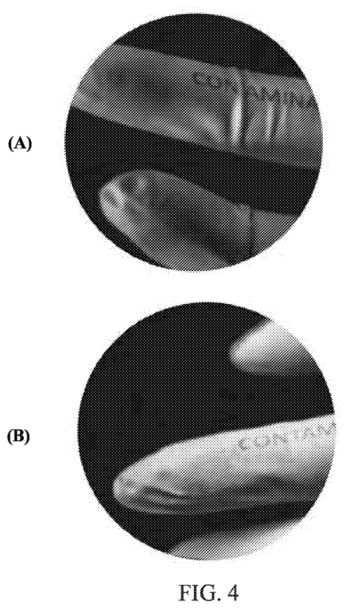

Another example of a silk-based ink composition printed on a substrate for sensing environmental stimuli is shown in FIG. 4. FIG. 4 panel (A) an ink composition that contains a molecule for sensing contamination, printed on a nitrile glove. Panel (B) shows a local demonstration of color change in response to the presence of contamination.

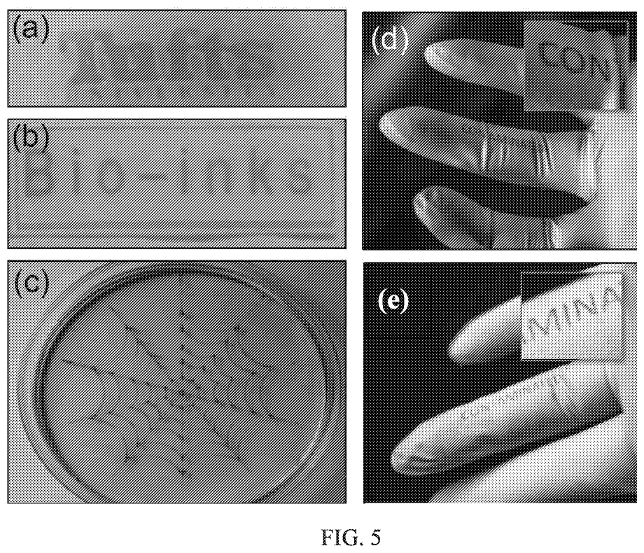

Substrates used to print ink compositions containing molecules for sensing environmental stimuli can vary. FIG. 5 shows examples of ink compositions for sensing environmental stimuli, printed on different surfaces. Panel (A) shows antibiotic inks printed on a bacterial lawn. Panel (B) shows enzyme inks on paper. Panel (C) shows bone deposition in predetermined geometries on a petri dish based on a pattern of ink containing growth factor such as BMP-8 (bone morphogenic protein) on the petri dish. Panel (D) shows ink compositions printed on a nitrile lab glove that initially are blue inks and change color to red as shown in Panel (E) when the nitrile glove is exposed to bacteria.

Any citations to publications, patents, or patent applications herein are incorporated by reference in their entirety. Any numerals used in this application with or without about/approximately are meant to cover any normal fluctuations appreciated by one of ordinary skill in the relevant art.

Other features, objects, and advantages of the present disclosure are apparent in the detailed description that follows. It should be understood, however, that the detailed description, while indicating aspects of the present disclosure, is given by way of illustration only, not limitation. Various changes and modifications within the scope of the invention will become apparent to those skilled in the art from the detailed description.

1. A biologically-based ink composition comprising:
a silk fibroin solution having a concentration of silk fibroin between 0.1 wt % and 10 wt %; and
a thickening agent and a humectant dispersed throughout the composition, wherein the thickening agent comprises a polysaccharide.

2. The biologically-based ink composition of claim 1, wherein the thickening agent and the humectant are present in the composition in a weight ratio of between 4:1 and 1:4.

3. The biologically-based ink composition of claim 1 or 2, wherein the thickening agent and the humectant are present in the composition in a weight ratio of between 2:1 and 1:2.

4. The biologically-based ink composition of any of the preceding claims, wherein the concentration of thickening agent in the composition is between 0.1 wt % and 10 wt %.

5. The biologically-based ink composition according to the immediately preceding claim, wherein the concentration of thickening agent in the composition is between 0.1 wt % and 4 wt %.

6. The biologically-based ink composition of any one of the preceding claims, wherein the concentration of humectant in the composition is between 0.1 wt % and 10 wt %.

7. The biologically-based ink composition of any one of the preceding claims, wherein the viscosity of the composition is between 1,000 cP and 10,000 cP at 23° C.

8. The biologically-based ink composition of the immediately preceding claim, wherein the viscosity of the composition is 3,000 cP to 5,000 cP measured at 23° C.

9. The biologically-based ink composition of the immediately preceding claim, wherein the composition has a viscosity between 1,000 cP and 2,500 cP when extruded or printed at a shear rate of 100 to 200 s⁻¹.

10. The biologically-based ink composition of any one of the preceding claims, wherein the polysaccharide is selected from the group consisting of an algin, an alginate, a guaran, a chitosan, a cellulose, and an arabinoxylan.

11. The biologically-based ink composition of any one of the preceding claims, wherein the polysaccharide is sodium alginate.

12. The biologically-based ink composition of any one of the preceding claims, wherein the humectant is glycerol.

13. The biologically-based ink composition of any one of the preceding claims, wherein the silk fibroin solution is an aqueous silk fibroin solution substantially free of organic solvent.

14. The biologically-based ink composition of any one of the preceding claims, wherein the silk fibroin has a molecular weight of about 3.5 kD to about 350 kD.

15. The biologically-based ink composition of any one of the preceding claims, wherein the silk fibroin solution further comprises one or more additives.

16. The biologically-based ink composition of any one of the preceding claims, wherein the one or more additives comprises a sensing agent.

17. The biologically-based ink composition of the immediately preceding claims, wherein the sensing agent comprises a temperature sensitive agent, a pH sensitive agent, a pressure sensitive agent, a light sensitive agent, a potentiometric sensitive agent, a chemi-sensitive agent, and combinations thereof.

18. The biologically-based ink composition of any one of the preceding claims, wherein the one or more additives comprises a conductive additive.

19. The biologically-based ink composition of any one of the preceding claims further comprising a biologically active agent.

20. The biologically-based ink composition of any one of the preceding claims further comprising a therapeutic agent.

21. An apparatus comprising
a substrate; and
a biologically-based ink composition adhered to a surface of the substrate, the biologically-based ink composition comprising a silk fibroin solution having a concentration of silk fibroin in a range of 0.1 wt % to 10 wt %; and a thickening agent and a humectant dispersed throughout the composition, wherein the thickening agent comprises a polysaccharide.

22. The apparatus of claim 21, wherein the substrate is characterized by its flexibility, such that when the printed article contacts an object it substantially conforms to the object's surface.

23. The apparatus of claim 21 or 22, wherein the substrate comprises a textile.

24. The apparatus of any one of the preceding claims, wherein the substrate is characterized as having pores, and wherein the biologically-based ink composition is adhered to at least a fraction of the pores.

25. The apparatus of claim 21, wherein the substrate is characterized as a rigid object.

26. The apparatus of any one of the preceding claims, wherein the biologically-based ink composition forms a pattern on the surface of the substrate.

27. The apparatus of the immediately preceding claim, wherein the biologically-based ink composition further comprises a sensing dye or agent, and wherein the pattern on the substrate forms a sensor.

28. The apparatus of any of the immediately two preceding claims, wherein the pattern spans across an entire surface of the substrate.

29. The apparatus of claim 26, wherein the pattern comprises one or more micro-channel.
